(19) United States
(12) Patent Application Publication
(10) Pub. No.: US 2013/0295115 A1
(43) Pub. Date: Nov. 7, 2013

(54) COMPOSITIONS AND METHODS FOR TREATING ALPHAVIRUS INFECTION

(71) Applicants: Sara Cherry, Philadelphia, PA (US); Patrick Rose, Parkville, MD (US)

(72) Inventors: Sara Cherry, Philadelphia, PA (US); Patrick Rose, Parkville, MD (US)

(21) Appl. No.: 13/885,536

(22) PCT Filed: Nov. 16, 2011
(86) PCT No.: PCT/US11/60952

§ 371 Date: Jul. 22, 2013

Related U.S. Application Data
(60) Provisional application No. 61/414,179, filed on Nov. 16, 2010.

(51) Int. Cl. A61K 31/713; G01N 33/50; C12Q 1/68; A61K 33/26

(52) U.S. Cl. CPC A61K 31/713; A61K 33/26; G01N 33/502; C12Q 1/6876
USPC 424/172.1; 514/44 R; 514/44 A; 514/1.2; 514/502; 536/23.1; 536/24.5; 530/389.1; 556/147; 424/184.1; 435/5; 435/6.12

(57) ABSTRACT

Provided herein are compositions and methods for treating a disease or disorder associated with an alphavirus infection. Specifically, the invention relates to administering a Natural Resistance-Associated Macrophage Protein (NRAMP) antagonist to prevent binding or infection of an alphavirus to its host.

### CROSS REFERENCE TO RELATED APPLICATIONS

This application claims priority to U.S. Provisional Patent Application 61/414,179, filed Nov. 16, 2010, which is incorporated by reference herein in its entirety.

### STATEMENT OF GOVERNMENT INTEREST

The work described herein was, in part, supported by National Institute of Health grants T32-AI055400, F32-AI078666, RO1AI079451 and U54AI057168. The United States government may have certain rights in this application.

### FIELD OF THE INVENTION

Compositions and methods are provided for treating a disease or disorder associated with an alphavirus infection. Specifically, embodiments of the invention relate to administering a Natural Resistance-Associated Macrophage Protein (NRAMP) antagonist to prevent binding or infection of an alphavirus to its host.

### BACKGROUND OF THE INVENTION

Arboviruses are a class of emerging and re-emerging pathogens that are transmitted to vertebrates from insects. Alphaviruses are a large family of arboviruses, which infect more than one million people annually, and have a broad host range in nature. Little is known about the host factor requirements for this class of viruses in either the insect or vertebrate, and no entry receptors have been identified for any arbovirus in part due to the lack of non-permissive cells. There are many alphaviruses distributed around the world with the ability to cause human disease. Infectious arthritis, encephalitis, rashes and fever being the most commonly observed.

Therefore, there exists a need, inter alia, for compositions and methods for treating an alphavirus infection, as well as for methods for identifying compositions that treat an alphavirus infection. The present invention addresses this need by regulating a target, a divalent metal ion transporter, Natural Resistance-Associated Macrophage Protein (NRAMP) used by alphaviruses in their infection cycle, and which can be exploited for genetic, drug-based or other known forms of therapy against alphaviruses.

### SUMMARY OF THE INVENTION

In one aspect, embodiments of the present invention provide methods for treating a disease or disorder associated with an alphavirus infection in a subject, the methods include: administering to the subject an effective amount of a molecule that inhibits the activity of a Natural Resistance-Associated Macrophage Protein (NRAMP), thereby treating the disease or disorder in the subject. In certain embodiments, the alphavirus is a Sindbis virus (SINV). In other embodiments, the alphavirus is a Venezuelan Equine Encephalitis virus.

In another aspect, embodiments of the present invention provide methods for identifying a molecule that treats a disease or disorder associated with an alphavirus infection, the methods include: contacting a cell that expresses a Natural Resistance-Associated Macrophage Protein (NRAMP) with a candidate molecule, and comparing the biological activity of the NRAMP in the cell contacted by the candidate molecule with the level of biological activity in a control cell not contacted by the candidate molecule, wherein an alteration in the biological activity of the NRAMP identifies the candidate molecule as a candidate molecule that treats a disease or disorder associated with the alphavirus infection.

In yet another aspect, embodiments of the present invention provide compositions, the compositions include: a molecule that inhibits the activity of a Natural Resistance-Associated Macrophage Protein (NRAMP), wherein said molecule is present in an amount effective to treat a disease or disorder associated with an alphavirus infection.

In a further aspect, embodiments of the present invention provide vaccines, the vaccines include: a molecule that inhibits the activity of a Natural Resistance-Associated Macrophage Protein (NRAMP), wherein said molecule is present in an amount effective to treat a disease or disorder associated with an alphavirus infection.

In a further aspect, embodiments of the present invention provide methods for predicting a risk for a disease or disorder associated with an alphavirus infection, in a subject, the methods include: testing a sample from a subject to detect the presence or absence of a Natural Resistance-Associated Macrophage Protein (NRAMP), wherein the presence of said NRAMP indicates the risk for said disease or disorder, thereby predicting said risk in said subject. In certain embodiments, the methods further include the step of obtaining the biological sample from the subject.

In yet another aspect, methods for increasing the expression of Natural Resistance-Associated Macrophage Protein (NRAMP) or fragment thereof in a subject and/or cell are provided, comprising the step of administering to the subject and/or cell a nucleic acid encoding said NRAMP or fragment thereof, thereby enhancing the ability of an alphavirus (e.g., a chimeric sindbis virus gene-based vaccine) to infect said subject and/or cell.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description. It is also contemplated that whenever appropriate, any embodiment of the present invention can be combined with one or more other embodiments of the present invention, even though the embodiments are described under different aspects of the present invention.

### BRIEF DESCRIPTION OF THE DRAWINGS

FIG. 1 shows that Sindbis virus productively infects Drosophila cells. (A). Supernatant from infected Drosophila cells (Top) was used to infect mammalian BHK cells (Bottom). (B) Drosophila or mammalian BHK cells were mock or ammonium chloride (NH4CI) treated and challenged with SINV. (C) Drosophila cells were pre-treated with the indicated dsRNAs and challenged with SINV. A representative experiment is shown and triplicate experiments of the mean±s.d of the Z-scores is graphed (Z<−2 is p<0.05) (virus, green; nuclei, blue).

FIG. 2 shows genome-wide RNAi screen and bioinformatics. (A) schematic of the RNAi screen. (B) Fraction of candidates that have homologs in indicated genera. Significant (p<0.001) enrichment of conserved genes and under-enrichment of Drosophila-only genes (p<0.001) demonstrated by chi-squared test comparing the observed fraction of genes to the fraction of the Drosophila genome as a whole. (C) Bioinformatic classification of candidates into biological process categories. Categories found enriched (p>0.01) relative to the whole gene set with five or more members are displayed. Processes highlighted in red were associated with vesicular trafficking.

FIG. 3. Cellular map of SINV screen candidates. All of the validated genes were classified according to sub-cellular compartments and cellular processes using annotation information from Gene Ontology, PANTHER, InterPro, and literature curation. Red genes are factors that facilitate infection while green factors restrict infection. Genes conserved in humans have an asterisk and the ones in black are the additional components of a complex identified in the primary screen but not re-tested.

FIG. 4 shows Drosophila NRAMP is required for binding and entry of Sindbis virus. Independent dsRNA against dNRAMP, the vATPase component vha26, the SINV reporter GFP and the negative control luciferase were challenged with (A) Sindbis virus-GFP(HRsp) in DL1 cells (B) Sindbis virus-GFP(HRsp) in Kc167 cells (C) Sindbis virus-GFP (dsTE12H) in DL1 cells. (D) Sindbis-GFP(S.A.AR86) in DL1 cells. (E) Wild type WNV detected using anti-E in DL1 cells. a-e (virus, green; nuclei, blue) (F) Cells pre-treated with the indicated dsRNA were either infected (red) or transfected with vRNA (blue) and triplicate mean±s.d. shown; *p<0.05. (G) Cells were pre-treated with dsRNA against dNRAMP or control dsRNA and bound with biotinylated Sindbis virus (red, virus; blue, nucleus). (H) The percentage of cells that had at least one viral particle bound is graphed with mean±s.d. for triplicate experiments; *p<0.05.

FIG. 5 shows that dNRAMP is required for Sindbis infection of adult flies and that virus infection is sensitive to iron treatment in both Drosophila and mosquito cells. (A) Survival curve of SINV challenged flies compared to control. (B) Viral titers of flies challenged with the indicated SINV concentrations or control at 6 days post infection. Mean±S.D. of three experiments. (C) Sindbis virus titers from flies of the indicated genotypes at either 3 (gray) or 6 (black) days post infection. Mean±S.D. of three experiments; *p<0.05. (D) VSV virus titers from flies of the indicated genotypes at either 3 (gray) or 6 (black) days post infection; Mean±S.D. of three experiments. Sindbis virus infection is also sensitive to iron treatment in both Drosophila and mosquito cells. E-G. Iron treatment of Drosophila DL1 cells (black) or Aedes aegypti Aag-2 cells (gray) challenged with (E) Sindbis virus (HRsp), (F) Sindbis virus (S.A.AR86), or (G) VSV. Mean±S.D. of three experiments; * p<0.05.

FIG. 6. Shows that NRAMP is conserved. (A) Boot-strapped (100-iterations) phylogenetic tree indicating the relationship between NRAMP of the indicated species. (B) Identity (red) and Similarity (yellow) Matrix indicating conservation between various species of NRAMP. (C) Identity (red) and Similarity (yellow) Matrix indicating conservation between dNRAMP and hNRAMP2 extracellular loops.

FIG. 7 shows that NRAMP2 expression is attenuated by iron treatment which can block Sindbis virus infection and binding (A) RT-PCR of hNRAMP1, hNRAMP2 and gAPDH in human U2OS cells in the presence or absence of iron. (B) U2OS cells challenged with SINV or VSV in the presence or absence of iron. A representative experiment is shown (green, virus; blue, nuclei). (C) 293T cells challenged with Sindbis virus (HRsp) in the presence or absence of iron. Mean±s.d of three experiments; * p<0.05. (D) MEFs challenged with Sindbis virus (HRsp) in the presence or absence of iron. Mean±s.d of three experiments; * p<0.05. (E) A representative image is shown of U2OS hNRAMP2 transduced cells either untreated or iron treated and bound with biotinylated SINV or VSV (red, virus; blue, nuclei).

FIG. 8 NRAMP2 is required for Sindbis virus and VEE infection of mammals. (A) U2OS cells challenged with the indicated virus in the presence or absence of iron. Mean±s.d. of three experiments; * p<0.05. (B) U2OS cells transfected with hNRAMP2 or the control vector (mock) were challenged with the indicated virus. Mean±s.d. of three experiments; *p<0.05. (C) A representative image is shown of U2OS hNRAMP2 transduced cells either untreated or iron treated and bound with biotinylated SINV or VSV (red, virus; blue, nucleus). (D) The percentage of cells that had at least one viral particle bound is graphed for the indicated virus with mean±s.d. for triplicate experiments shown; *p<0.05. (E) Mock or hNRAMP2-transfected U2OS cells were either untreated or iron treated and bound with biotinylated Sindbis virus, immunoprecipitated and visualized by immunoblot. Left panel shows that cells that were uninfected do not have a band while input labeled virus is visualized as a 60 kD band. (F) NRAMP2^{fl/fl} cells were transduced with MSCV-Cre-ires-GFP or MSCV-ires-GFP retroviruses and challenged with the indicated virus. The percent infection of retrovirally transduced cells (GFP⁺) is shown for triplicate experiments mean±s.d.; *p<0.05. (G) Sindbis and Ross River chimeric viruses were used to challenge NRAMP2^{fl/fl} cells transduced with MSCV-Cre-ires-GFP or MSCV-ires-GFP retroviruses as indicated. Mean±s.d. of three experiments; * p<0.05. (gp, glycoproteins; nsp, nonstructural proteins) (H) U2OS cells challenged with VEE or CHIKV, in the presence or absence of iron; Mean±s.d. of three experiments; * p<0.05. (I) NRAMP2^{fl/fl} cells were transduced with MSCV-Cre-ires-GFP or MSCV-ires-GFP retroviruses and challenged with VEE or CHIKV and the percent infection of retrovirally transduced cells is shown for three experiments. Mean±s.d.; *p<0.05. (J) Drosophila cells pre-treated with dsRNA against dNRAMP, the viral reporter GFP or the negative control luciferase were challenged with VEE and the percent infection for three experiments shown Mean±s.d.; *p<0.05.

FIG. 9. NRAMP2 facilitates binding and infection of Sindbis virus in mammalian cells. U2OS cells were either untreated or iron treated and bound with biotinylated Sindbis virus, biotinylated VSV or biotinylated IgG, precipitated with streptavidin beads and visualized by immunoblot. GAPDH and NRAMP2 are observed in the input, whereas only Sindbis virus precipitates with NRAMP2; and only in the absence of iron treatment which reduces NRAMP2 levels. Representative of 5 experiments.

FIG. 10. NRAMP2′ cells express mNRAMP2, and can be induced to delete the floxed exons by Cre. (A) RT-PCR of mNRAMP1, mNRAMP2 and GAPDH in NRAMP2^{fl/fl} cells; (B) PCR of genomic DNA from NRAMP2^{fl/fl} cells transduced with MSCV-Cre iGFP or MSCV-iGFP using the indicated primers to monitor recombination.

### DETAILED DESCRIPTION OF THE INVENTION

Embodiments of the present invention relate to compositions and methods for treating a disease or disorder associated with an alphavirus infection. Specifically, embodiments of the present invention relate to administering an NRAMP antagonist to prevent binding or infection of an alphavirus to its host.

In one aspect, provided herein are methods for treating a disease or disorder associated with an alphavirus (e.g., Sindbis virus (SINV) or Venezuelan Equine Encephalitis (VEE) virus) infection in a subject, the methods include: administering to the subject an effective amount of a molecule that inhibits the activity of a Natural Resistance-Associated Macrophage Protein (NRAMP), thereby in treating the disease or disorder in the subject.

In another aspect, provided herein are methods for identifying a molecule that treats a disease or disorder associated with an alphavirus (e.g., Sindbis virus or Venezuelan Equine Encephalitis virus) infection, the methods include contacting a cell that expresses a Natural Resistance-Associated Macrophage Protein (NRAMP) with a candidate molecule, and comparing the biological activity of the NRAMP in the cell contacted by the candidate molecule with the level of biological activity in a control cell not contacted by the candidate molecule, wherein an alteration in the biological activity of the NRAMP identifies the candidate molecule as a candidate molecule that treats a disease or disorder associated with the alphavirus infection.

In yet another aspect, provided herein are compositions (e.g., vaccines) comprising: a molecule that inhibits the activity of a Natural Resistance-Associated Macrophage Protein (NRAMP), wherein the molecule is present in an amount effective to treat a disease or disorder associated with an alphavirus (e.g., Sindbis virus or Venezuelan Equine Encephalitis virus) infection.

In a further aspect, provided herein are methods for predicting a risk for a disease or disorder associated with an alphavirus (e.g., Sindbis virus or Venezuelan Equine Encephalitis virus) infection, in a subject, the methods include: testing a sample from a subject to detect the presence or absence of a Natural Resistance-Associated Macrophage Protein (NRAMP), wherein the presence of said NRAMP indicates the risk for the disease or disorder, thereby predicting the risk in said subject. In certain embodiments, the methods further include the step of obtaining the biological sample from the subject.

The inventors of the instant application surprisingly and unexpectedly found that NRAMP is required for an alphavirus to bind and infect its host. Accordingly, the inventors found that inhibiting NRAMP can prevent binding or infection of an alphavirus to its host.

The term “alphavirus,” as used herein, refers to any alphavirus, including but not limited to, Sindbis virus, Venezuelan Equine Encephalitis virus, Semliki Forest Virus, Western equine encephalitis virus, Eastern equine encephalitis virus, Ross River virus, Semliki Forest Virus, and Barmah Forest virus.

As used herein, “NRAMP” refers to all isoforms of NRAMP. There are currently 2 known isoforms of NRAMP 1 and 2.

In certain embodiments of the present invention, the NRAMP1 and 2 includes any of the known isoforms, or variants thereof available in the art, including, but not limited to, those listed in Genbank, for example, accession numbers: AAC59756, AAX21761, AAK43768, AAD51941, AAK43832, ABH88063, NP_{—}998986, CAA57541, BAA07370, NP_{—}038640, BAA08908, and CAD55951.

In certain embodiments of the present invention, the NRAMP 2 includes any of the known isoforms, or variants thereof available in the art, including, but not limited to, those listed in Genbank, for example, accession numbers: AAC24496, ABV00878, ACS34965, AAF71821, AAC21459, AAC18078, AAC53319, and NP_{—}001139633.

As used herein, a “NRAMP antagonist” is any molecule that is able to decrease the amount or activity of NRAMP 1 or 2, either within a cell or within a physiological system. For example, a NRAMP antagonist may be a molecule that inhibits expression of NRAMP at the level of transcription, translation, processing, or transport; it may affect the stability of NRAMP or conversion of the precursor molecule to the active, mature form; it may affect the neutralization of NRAMP or the ability of NRAMP to bind to one or more receptors; or it may interfere with NRAMP signaling.

In some embodiments according to the present invention, the NRAMP antagonist will be an iron containing compound or composition. In some embodiments, the iron containing compound or composition is an iron supplement. Iron supplements are well known in the art and are available both in prescription form as a dietary supplement. Iron supplements are available for oral and/or parenteral administration. Any of the foregoing iron containing compositions is contemplated for use with methods of the present invention. For example, without limitation, iron supplements will be administered to treat a subject with an alphavirus (e.g., Sindbis virus or Venezuelan Equine Encephalitis virus) infection. In another example, without limitation, administration will be administered to prevent a subject from being infected with an alphavirus (e.g., Sindbis virus or Venezuelan Equine Encephalitis virus). In some embodiments, an iron containing composition will be used in addition to one or more other NRAMP antagonists.

In certain embodiments, the NRAMP antagonist is an oligonucleotide agent. As used herein, an “oligonucleotide agent” refers to a single stranded oligomer or polymer of ribonucleic acid (RNA) or deoxyribonucleic acid (DNA) or both or modifications thereof, which is antisense with respect to its target. This term includes oligonucleotides composed of naturally-occurring nucleobases, sugars and covalent internucleoside (backbone) linkages as well as oligonucleotides having non-naturally-occurring portions which function similarly.

Oligonucleotide agents include both nucleic acid targeting (NAT) oligonucleotide agents and protein-targeting (PT) oligonucleotide agents. NAT and PT oligonucleotide agents refer to single stranded oligomers or polymers of ribonucleic acid (RNA) or deoxyribonucleic acid (DNA) or both or modifications thereof. NATs designed to bind to specific RNA or DNA targets have substantial complementarity, e.g., at least 70, 80, 90, or 100% complementary, with at least 10, 20, or 30 or more bases of a target nucleic acid, and include antisense RNAs, microRNAs, antagomirs and other non-duplex structures which can modulate expression. The NAT oligonucleotide agents can target any nucleic acid, e.g., a miRNA, a pre-miRNA, a pre mRNA, an mRNA, or a DNA. These NAT oligonucleotide agents may or may not bind via Watson-Crick complementarity to their targets. PT oligonucleotide agents bind to protein targets, preferably by virtue of three-dimensional interactions, and modulate protein activity. They include decoy RNAs, aptamers, and the like.

In certain embodiments, the oligonucleotide agent is a double-stranded ribonucleic acid (dsRNA) molecule packaged in an association complex, such as a liposome, for inhibiting the expression of a gene (e.g., NRAMP) in a cell or mammal, wherein the dsRNA comprises an antisense strand comprising a region of complementarity which is complementary to at least a part of an mRNA formed in the expression of the gene, and wherein the region of complementarity is less than 30 nucleotides in length, generally 19-24 nucleotides in length, and wherein said dsRNA, upon contact with a cell expressing said gene, inhibits the expression of said gene. The dsRNA comprises two RNA strands that are sufficiently complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of a gene, the other strand (the sense strand) comprises a region which is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s). The dsRNA can be synthesized by standard methods known in the art as further discussed below, e.g., by use of an automated DNA synthesizer, such as are commercially available from, for example, Biosearch, Applied Biosystems, Inc.

The dsRNAs suitable for packaging in the association complexes described herein can include a duplex structure of between 18 and 25 basepairs (e.g., 21 base pairs). In some embodiments, the dsRNAs include at least one strand that is at least 21 nucleotides long. In other embodiments, the dsRNAs include at least one strand that is at least 15, 16, 17, 18, 19, 20, or more contiguous nucleotides.

Examples of other oligonucleotide molecules include, for example, but are not limited to, miRNA, siRNA, anti-sense RNA, enzymatic RNA, and aptamer. These molecules are well known and fully described in U.S. Patent Publication 20100196354, which is incorporated by reference herein in its entirety.

In some embodiments, the NRAMP antagonist is an antibody that inhibits NRAMP activity, or fragments thereof such as Fab, Fab′, F(ab′)₂ fragments, Fv fragments, single chain antibodies and other forms of “antibodies” that retain the ability to bind to NRAMP. In certain embodiments, the antibody is a pan-specific anti-NRAMP antibody. In one example, the pan-specific anti-NRAMP antibody binds to and neutralizes one or more isoforms of NRAMP. In another example, the pan-specific anti-NRAMP antibody blocks or inhibits the binding of a protein to one or more isoforms of NRAMP.

In some embodiments, the antibody is a monoclonal antibody. In other embodiments, the antibody is a human antibody. In yet other embodiments, the antibody is a humanized antibody. In further embodiments, the antibody is a chimeric antibody. In particular embodiments, the antibody is a humanized form of a murine monoclonal antibody.

Antibodies also include polypeptides with amino acid sequences substantially similar to the amino acid sequence of the variable or hypervariable regions of the heavy and/or light chain. Substantially the same amino acid sequence is defined herein as a sequence with at least 70%, 75%, 80%, 85%, 90%, 95%, or 99% identity to a compared amino acid sequence, as determined by the FASTA search method in accordance with Pearson and Lipman, Proc. Natl. Acad. Sci. USA 85:2444-2448 (1988).

The antibodies of embodiments of the present invention are specific for NRAMP. Antibody specificity refers to selective recognition of an antibody for a particular epitope of an antigen. The antibodies may exhibit both species and molecule selectivity, or may be selective with respect to molecule only and bind to NRAMP of more than one species (e.g., antibodies may bind to human, murine, rat, dog and/or rabbit NRAMP). In certain preferred embodiments, the antibodies bind to human NRAMP. Whether an antibody binds specifically to a NRAMP can be determined, e.g., by a binding assay such as an ELISA, employing a panel of antigens.

Specificity of the antibodies can be determined based on affinity and/or avidity. Affinity, represented by the equilibrium constant for the dissociation of an antigen with an antibody (K_{d}), measures the binding strength between an antigenic determinant and an antibody-binding site. Avidity is the measure of the strength of binding between an antibody with its antigen. Avidity is related to both the affinity between an epitope with its antigen binding site on the antibody, and the valence of the antibody, which refers to the number of antigen binding sites of a particular epitope. Antibodies typically bind with a dissociation constant (K_{d}) of about 10⁻⁵ to about 10⁻¹¹ liters/mol (e.g., K_{d}<100 nM). Any K_{d} greater than about 10⁻⁴ liters/mol is generally considered to indicate nonspecific binding. The lesser the value of the K_{d}, the stronger the binding strength between an antigenic determinant and the antibody binding site.

The antibodies of the invention bind to NRAMP with a Kd of preferably about 1×10⁻⁸ M⁻¹ or less, more preferably about 1×10⁻⁹ M⁻¹ or less, more preferably about 1×10⁻¹° M⁻¹ or less, and most preferably about 1×10⁻¹¹ M⁻¹ or less.

Antibodies of the present invention can be monospecific, bispecific or multispecific. Monospecific antibodies bind to only one antigen. Bispecific antibodies (BsAbs) are antibodies that have two different antigen-binding specificities or sites. Multispecific antibodies have more than two different antigen-binding specificities or sites. Where an antibody has more than one specificity, the recognized epitopes can be associated with a single antigen or with more than one antigen.

Embodiments of the present invention also include nucleic acid molecules that encode an anti-NRAMP antibody or portion thereof. Nucleic acids may encode an antibody heavy chain, comprising any one of the VH regions or a portion thereof, or any one of the VH CDRs, including any variants thereof, as disclosed herein. Embodiments of the present invention also include nucleic acid molecules that encode an antibody light chain comprising any one of the VL regions or a portion thereof or any one of the VL CDRs, including any variants thereof as disclosed herein. In certain embodiments, the nucleic acid encodes both a heavy and light chain, or portions thereof.

Embodiments of the present invention also include recombinant vectors comprising any of the nucleic acid molecules described herein. The vector may comprise a nucleic acid of nucleotide or polypeptide sequences or a nucleic acid encoding antibody chains or portions thereof.

Exemplary vectors include plasmids, phagemids, cosmids, viruses and phage nucleic acids or other nucleic acid molecules that are capable of replication in a prokaryotic or eukaryotic host. The vectors typically contain a marker to provide a phenotypic trait for selection of transformed hosts such as conferring resistance to antibiotics such as ampicillin or neomycin

The vector may be an expression vector, wherein the nucleic acid encoding the NRAMP antagonist is operably linked to an expression control sequence. Typical expression vectors contain transcription and translation terminators, initiation sequences, and promoters useful for regulation of the expression of the nucleic acid molecules of the invention. The vectors may also contain genetic expression cassettes containing an independent terminator sequence, sequences permitting replication of the vector in both eukaryotes and prokaryotes, i.e., shuttle vectors and selection markers for both prokaryotic and eukaryotic systems. When the vector contains nucleic acids encoding both a heavy and light chain or portions thereof, the nucleic acid encoding the heavy chain may be under the same or a separate promoter. The separate promoters may be identical or may be different types of promoters.

Suitable promoters include constitutive promoters and inducible promoters. Representative expression control sequences/promoters include the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha mating factors, promoters derived from the human cytomegalovirus, metallothionine promoter, murine mammary tumor virus promoter, Rous sarcoma virus promoter, polyhedrin promoter and promoters derived from polyoma, adenovirus, retrovirus, and simian virus, e.g., the early and late promoters of SV40.

Embodiments of the present invention also include non-human hosts such as cells or organisms containing a nucleic acid molecule or a vector of the invention. By “host” it is meant a non-human unicellular or multicellular organism or a “host cell”, which refers to a cell or population of cells into which a nucleic acid molecule or vector according to embodiments of the present invention is introduced. “A population of host cells” refers to a group of cultured cells into which a nucleic acid molecule or vector according to embodiments of the present invention can be introduced and expressed.

A host of the present invention may be prokaryotic or eukaryotic. Suitable prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces. Suitable eukaryotic cells include yeast and other fungi, insect cells, plant cells, human cells, and animal cells, including mammalian cells, such as hybridoma lines, COS cells, NS0 cells and CHO cells.

Embodiments of the present invention also include methods of producing an antibody of the present invention, which entails culturing a host cell expressing one or more nucleic acid sequences encoding an antibody of the present invention, and recovering the antibody from the culture medium. In certain embodiments, the antibody is purified by separating it from the culture medium. Antibodies comprising more than one chain can be produced by expressing each chain together in the same host; or as separate chains, which are assembled before or after recovery from the culture medium.

Embodiments of the present invention also include methods of producing a NRAMP antagonist molecule, the method comprising: culturing a host cell; and recovering the NRAMP antagonist molecule from said host cell.

Depending on the expression system and host selected, the molecules are produced by growing host cells transformed by an expression vector described above whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art. For example, once expressed, the product may be isolated and purified by any number of techniques, well known in the art. A protein according to embodiments of the present invention obtained as above may be isolated from the interior or exterior (e.g., medium) of the cells or hosts, and purified as a substantially pure homogeneous protein. The method for protein isolation and purification is not limited to any specific method. In fact, any standard method may be used. For instance, column chromatography, filtration, ultrafiltration, salt precipitation, solvent precipitation, solvent extraction, distillation, immunoprecipitation, SDS-polyacrylamide gel electrophoresis, isoelectric point electrophoresis, dialysis, and recrystallization may be appropriately selected and combined to isolate and purify the protein.

For chromatography, for example, affinity chromatography, ion-exchange chromatography, hydrophobic chromatography, gel filtration, reverse phase chromatography, adsorption chromatography, and such may be used (ed. Daniel R. Marshak et al. (1996) Strategies for Protein Purification and Characterization: A Laboratory Course Manual., Cold Spring Harbor Laboratory Press). These chromatographies may be performed by liquid chromatography, such as, HPLC and FPLC. Thus, embodiments of the present invention provide highly purified proteins produced by the above methods.

Embodiments of the present invention also provide a pharmaceutical composition comprising the peptide, nucleic acid, vector, or host cell of embodiments of this invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or more therapeutic agents.

Embodiments of the present invention also provide a pharmaceutical composition comprising an iron containing compound or composition and one or more pharmaceutically acceptable carriers. The pharmaceutical composition may include one or more therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions according to embodiments of the present invention may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Exemplary pharmaceutically acceptable carriers include, but are not limited to, 0.01-0.1 M and preferably 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in US Appl. Publ. No. 2002/0102208 A1, which is incorporated herein by reference in its entirety. Such articles of manufacture will preferably have labels or package inserts indicating that the associated compositions are useful for treating a subject suffering from, or predisposed to an alphavirus infection.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Embodiments of the present invention further provide methods of treating a disease or condition, comprising administering to a mammal in need thereof a therapeutically effective amount of a NRAMP antagonist molecule.

In one embodiment, provided herein is a method for treating a disease or disorder associated with an alphavirus infection, the method comprising: administering to said subject an effective amount of a molecule that inhibits the activity of a Natural Resistance-Associated Macrophage Protein (NRAMP), thereby in treating said disease or disorder in said subject. In one embodiment, the alphavirus is a Sindbis virus. In another embodiment, the alphavirus is a Venezuelan Equine Encephalitis virus.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented. In a particular embodiment, treatment may refer to preventing the infection of an alphavirus to its host.

Examples of disease or disorder caused by or otherwise associated with an alphavirus infection include, but are not limited to, Sindbis fever, Venezuelan equine encephalitis or encephalomyelitis, encephalitis, polyarthritis, rash and fever.

The NRAMP antagonist may be administered alone, or in combination with one or more therapeutically effective agents or treatments. The other therapeutically effective agent may be conjugated to NRAMP antagonist, incorporated into the same composition as NRAMP antagonist, or may be administered as a separate composition. The other therapeutically agent or treatment may be administered prior to, during and/or after the administration of the NRAMP antagonist. In some embodiments, the treatment further employs use of an additional and synergistic treatment method.

The administration of an NRAMP antagonist with other agents and/or treatments may occur simultaneously, or separately, via the same or different route, at the same or different times. Dosage regimens may be adjusted to provide the optimum desired response (e.g., a therapeutic or prophylactic response).

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

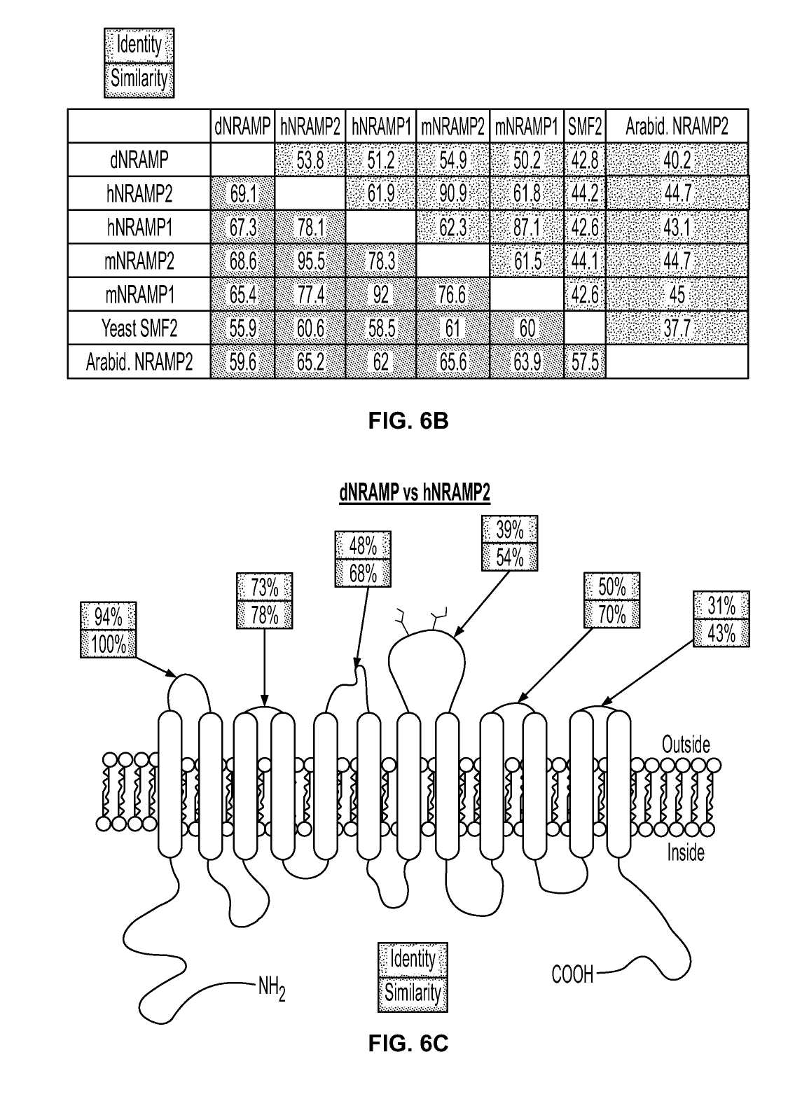

In some cases, it will be desirable, for example, for alphavirus based gene-therapy and vaccine delivery, to enhance the ability of an alphavirus to infect a cell and/or a subject. In such a case it will be advantageous to increase the expression in the subject or cell of an NRAMP and or a fragment thereof that facilitates alphavirus entry. For example, over-expression of the receptor may be useful as adjuvant for chimeric sindbis virus gene-based vaccine delivery. Thus, in certain embodiments of the present invention methods are provided for increasing the expression of Natural Resistance-Associated Macrophage Protein (NRAMP) or fragment thereof in a subject and/or cell are provided, comprising the step of administering to the subject and/or cell a nucleic acid encoding said NRAMP or fragment thereof, thereby enhancing the ability of an alphavirus (e.g., a chimeric sindbis virus gene-based vaccine) to infect said subject and/or cell. In some embodiments, the methods further comprise the step of administering an alphavirus based gene-therapy or vaccine. In some embodiments, NRAMP expression will be increased at least two fold. In some embodiments, the NRAMP fragment will include one or more of the extracellular loops, such as the first extracellular loop which is 94% identical between humans and Drosophila (FIG. 6C).

“Administration” to a subject is not limited to any particular delivery system and may include, without limitation, parenteral (including subcutaneous, intravenous, intramedullary, intraarticular, intramuscular, or intraperitoneal injection) rectal, topical, transdermal or oral (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described above). Once again, physiologically acceptable salt forms and standard pharmaceutical formulation techniques are well known to persons skilled in the art (see, for example, Remington's Pharmaceutical Sciences, Mack Publishing Co.).

Compositions according to embodiments of the present invention (e.g., NRAMP antagonist) may be administered parenterally (e.g., intravenous, subcutaneous, intraperitoneal, intramuscular). Further, the compositions according to embodiments of the present invention may be administered by intravenous infusion or injection. The compositions according to embodiments of the present invention may be administered by intramuscular or subcutaneous injection. In some embodiments, the compositions may be administered orally. As used herein, a “composition” refers to any composition that contains a pharmaceutically effective amount of a NRAMP antagonist.

The methods of treatment described herein can be used to treat any suitable mammal, including primates, such as monkeys and humans, horses, cows, cats, dogs, rabbits, and rodents such as rats and mice. In one embodiment, the mammal to be treated is human.

In another embodiment, the invention provides a method for predicting a risk for a disease or disorder associated with an alphavirus infection, in a subject, the method comprising testing a sample from the subject to detect the presence or absence of a Natural Resistance-Associated Macrophage Protein (NRAMP), wherein the presence of said NRAMP indicates the risk for said disease or disorder, thereby predicting said risk in said subject. In some embodiments, the methods further include the step of obtaining the biological sample from the subject. Nucleotide and protein based detection methods are well known in the art. Any suitable detection method, known to one of skilled in the art, can be used.

All sequence citations, accession numbers, references, patents, patent applications, scientific publications or other documents cited are hereby incorporated by reference.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLES

### Materials and Methods

### Cells and Virus and Reagents

DL1, KC167, Aag-2, C6/36, U2OS, 293T, MEFs were maintained as described in Methods. SINV and SINV-GFP(HRsp and dsTE12H) were generated in BHK cells and then propagated in C6/36 cells. VSV and VSV-GFP were propagated in BHK cells. WNV (WNV lineage I strain 3000.0259 NY 2000) virus was propagated in C6/36 cells. MSCV-iGFP and MSCV-CreiGFP retroviral supernatants were generated.

### Drosophila RNAi Screen

DL1 cells were seeded into 384-well plates pre-arrayed with 250 ng/well dsRNA (targeting 13,071 genes, obtained from Ambion, Inc.) and incubated for three days followed by infection with SIN-GFP. 36 hours post infection the plates were processed for microscopy, imaged and automated image analysis was used to calculate the percentage of infected cells. The entire screen was performed in duplicate.

### RNA Bypass

DL1 cells were seeded into 96-well plates pre-arrayed with 750 ng/well and incubated for three days. Next, the cells were either infected with SINV-GFP or transfected with SINV-GFP genomic viral RNA. After 36 hours the cells were processed for microscopy.

### Virus Binding Assays

Biotinylated SINV was bound to pre-chilled cells for 45 minutes at 4° C. Unbound virus was removed and cells were processed for microscopy using streptavidin-texas red or for biochemical analysis by immunoprecipitation followed by immunoblot with streptavidin-HRP.

mNRAMP2^{fl/fl} Assays

mNRAMP2^{fl/fl} MEFs were generated and immortalized with SV40 T-antigen. Cells were infected with either MSCViGFP or MSCVCre-iGFP and infected with either SINV HRsp (MOI=0.1) or VSV (MOI=0.1) and visualized using anti-SINV E1 or anti-VSV G 10 hours post infection.

Cells and Virus and Reagents.

DL4, KG167, and Aag-2 cells were maintained in Schneider's Medium (Invitrogen-GIBCO, Carlsbad, Calif.), supplemented with 10% heat inactivated Fetal Bovine Serum (Sigma) 100 U/mL of penicillin, 100 mg/ml of streptomycin (Invitrogen-GIBCO, Carlsbad, Calif.), and 100 mg/mL Hepes (Invitrogen-GIBCO, Carlsbad, Calif.). U205, 293T, and MEFs were maintained in DMEM (Invitrogen-GIBCO, Carlsbad, Calif.), supplemented with 10% heat inactivated Fetal Bovine Serum (Invitrogen-GIBCO, Carlsbad, Calif.), 100 U/mL of penicillin, 100 mg/mL of streptomycin (Invitrogen-GIBCO, Carlsbad, Calif.). Other chemicals were purchased from Sigma. Texas Red conjugated secondary antibodies were obtained from Jackson Immunochemicals. Streptavidin conjugates were obtained from Thermo Scientific.

Primers used for these studies:

| | |
|---|---|
| RT-PCR: | |
| Human NRAMP1 | |
| F: TCAAACTTCTCTGGGTGCTGCTCT | (SEQ ID NO: 1) |
| R: AGAGCAGATTGAATGCAATGGCCG | (SEQ ID NO: 2) |
| Human NRAMP2 | |
| F: AGGACATGCCCAGTGCAATCAAAC | (SEQ ID NO: 3) |
| R: TTACTTTGGCCCTGCTGCACTCTA | (SEQ ID NO: 4) |
| Mouse NRAMP1 | |
| F: TGTTGCCTCTTTGCTTGCTGGATG | (SEQ ID NO: 5) |
| R: ATGAGCAAAGCCATCACTTCGTGC | (SEQ ID NO: 6) |
| Mouse NRAMP2 | |
| F: CGACAAATGGCCTTTGCTGTCTGT | (SEQ ID NO: 7) |
| R: ATATAGGGTTGCAGATGGGTGGCA | (SEQ ID NO: 8) |
| Genomic mouse NRAMP2: | |
| F1: ATGGGCGAGtTAGAGGCttT | (SEQ ID NO: 9) |
| R1: CCTGCATGTCAGAACCAATG | (SEQ ID NO: 10) |
| F7-8: TAGCTTTCc6TGGTGTTTGG | (SEQ ID NO: 11) |
| R7-8: GAGCAGCCCAGAAGTAGGTG | (SEQ ID NO: 12) |
| R9: ATTTGGTGGTAAACACTGGC | (SEQ ID NO: 13) |

Sequences targeting NRAMP that were used for the dsRNA experiments in the Examples below:

(SEQ ID NO: 14)
TAATACGACTCACTATAGGTGGACAGCAGGTATATGGCAAATTAACCC
TCACTAAAGGTAGCAGCATGTGGAAACTCG
(SEQ ID NO: 15)
TAATACGACTCACTATAGGAAAGACAAGTCGATGTACGAATAATACG
ACTCACTATAGGTTACAGTCCATTAACAAACTCG

### Drosophila Whole Genome RNAi Screen

18,000 cells were seeded into 384-well plates pre-arrayed with 250 ng/well dsRNA (Ambion) in 10 μL of serum free media using automated liquid handling (Wellmate). One hour later 20 μL of complete media was added, and the plates were incubated for three days and then infected with SINV (MOI=10) for 36 hours. The plates were fixed in PBS/4% Formaldehyde for 10 minutes and washed twice in PBS. Cells were then stained with 5 μg/mL Hoechst 33432 and washed twice with PBS. GFP and DAPI images were captured, and 3 sites per well were imaged at 20× (ImageXpress Micro, Molecular Devices). Automated image analysis segmented the images and was used to determine the number of DAPI cells and the number of GFP cells. The percentage of infected cells was calculated, averaged for the three sites, and log transformed. The plate median was calculated as was the interquartile range. These metrics were used to calculate a robust Z score. Wells with <5 19 bp off-targets and a Z>2 or Z<−2 in duplicate (p<0.05) were the primary candidates. Cytotoxic candidates were determined by decreased nuclei, represented by a Robust Z score<−2 in duplicate wells; −30% decrease. Independent secondary amplicons were chosen from a different region within the target gene using SNAPdragon (Flybase.org) and used to generate dsRNA for secondary screening. Normalized average Z-scores were calculated and a one-sample student's t-test was used to evaluate if the mean of the average Z-scores of the secondary replicates were significant.

### Bioinformatics

Homologs were determined using homologene (NCBI) and a Chi-squared test was performed to determine significance. Gene Ontology enrichment was calculated using Cytoscape and redundant terms were eliminated. Phylogenetic analysis was done by aligning sequences with Clustal W and phylogentic tree was constructed using FLnjtree. Identity/Similarity Matrix of NRAMP sequences was done using MatGat 2.0.

RNAi vRNA Bypass

34,000 cells were seeded into 96-well plates pre-arrayed with 750 ng/well dsRNA in 30 μL of serum free media. One hour later 70 pL of complete media was added and the plates were incubated for three days. Next, the cells were infected with SINV (MOI=10), or transfected with 0.1 pg purified SINV genomic RNA using Effectene (Qiagen) following the manufacturer's protocol. 36 hours later the cells were fixed and processed for microscopy.

### Virus Biotinylation

SINV harvested from C6/36 cells was pre-cleared (1200 rpm for 4 minutes), and pelleted through a 25% sucrose cushion (SW32 @ 21,000 rpm, 4° C.) for 2 hours and resuspended in PBS. This purified virus was biotinylated with EZ-link Sulfo-NHS-LC-biotin (Thermo Scientific, Rockford, Ill.) for 30 minutes at room temperature and quenched with 1 mM Tris-Cl (pH 7.5).

### Adult Fly Infections

Flies were obtained from the Bloomington stock center and maintained on standard medium at room temperature. 4- to 7-day-old adult female flies were inoculated with SINV as previously described. Groups of at least 20 flies were challenged for mortality studies. Flies were processed for plaque assay by crushing and tittering on BHK cells.

### Iron Treatment

Cells were treated with 160 μM Ammonium Iron(III) Citrate for three days prior to and throughout the infection.

### Mammalian Transfections and Infections

U2OS cells were transfected with using Fugene per manufacturer's protocols. Cells were infected with SINV-GFP (MOI=0.1) or VSV-GFP (MOI−0.1) and fixed at 10 hours post-infection.

### RT-PCR

Total RNA was purified using Trizol and used for reverse transcription with random priming and MLV-RT (Invitrogen) followed by PCR using the indicated primers.

### Example 1

### Characterization of the Infection of Drosophila DL1 Cells with a Recombinant Sindbis Virus (HRsp) Expressing GFP from a Subgenomic Promoter

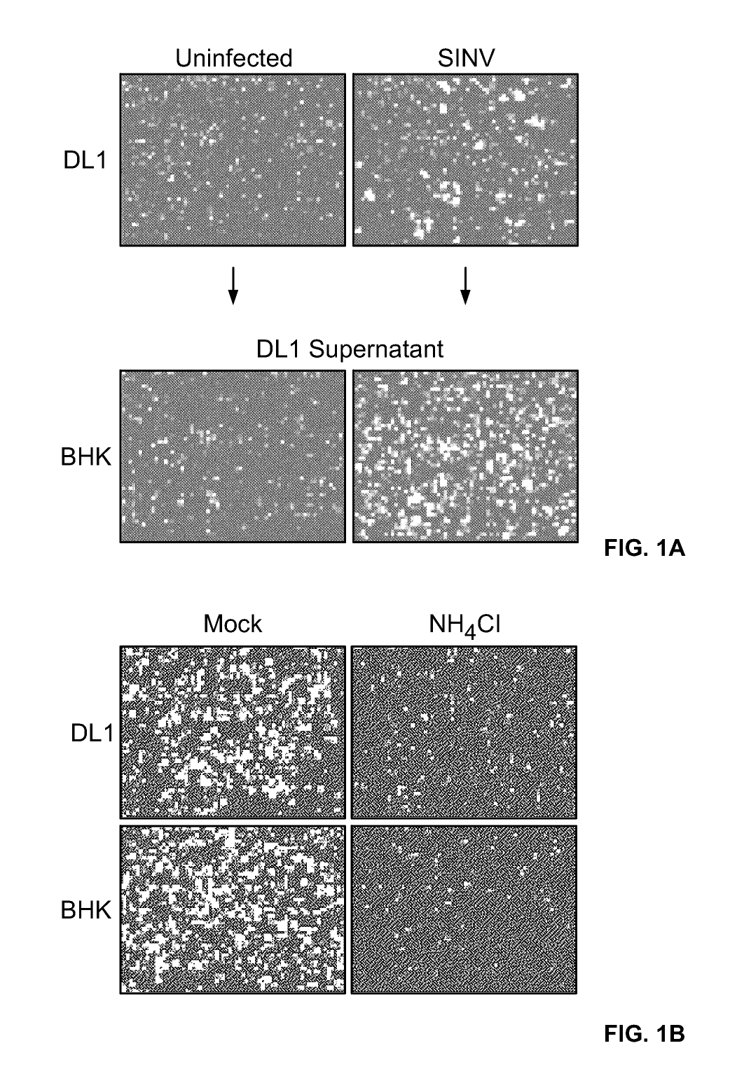

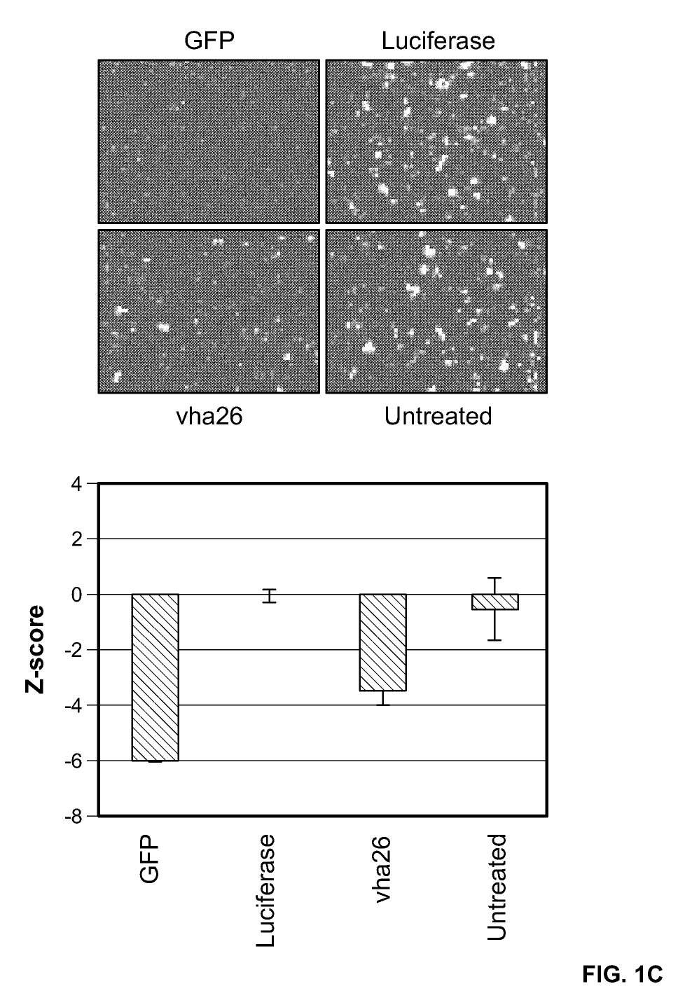

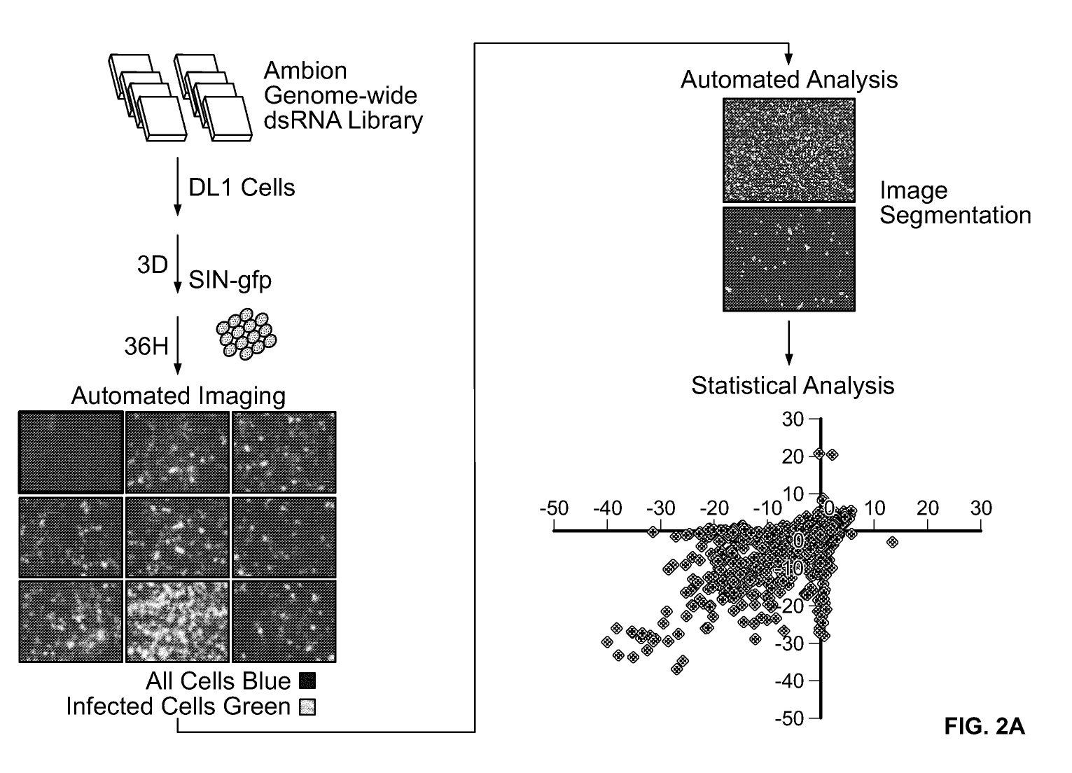

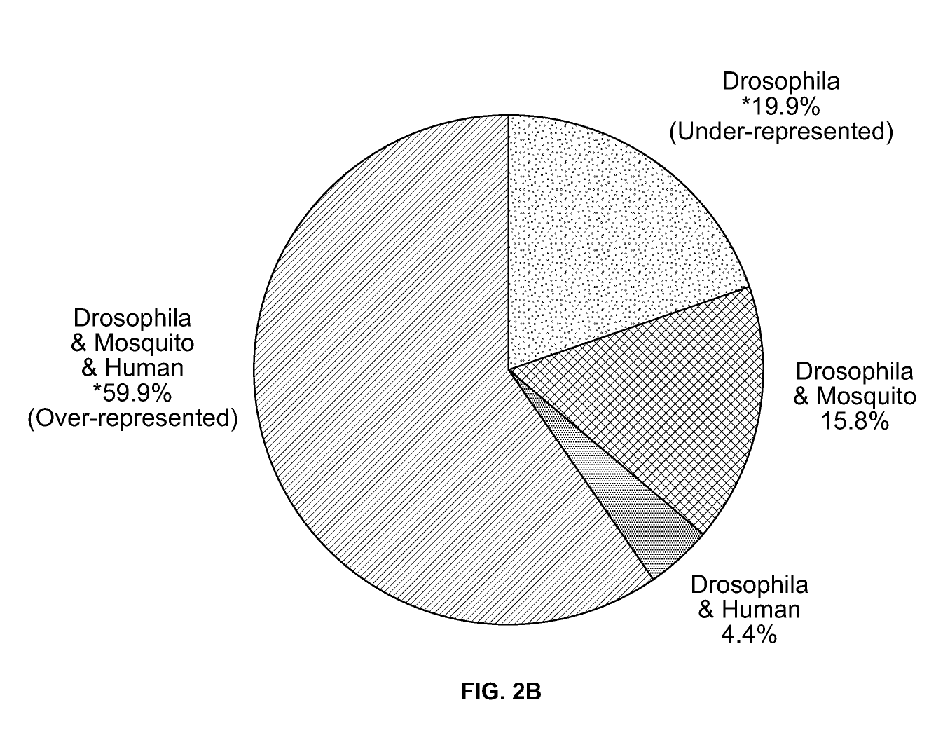

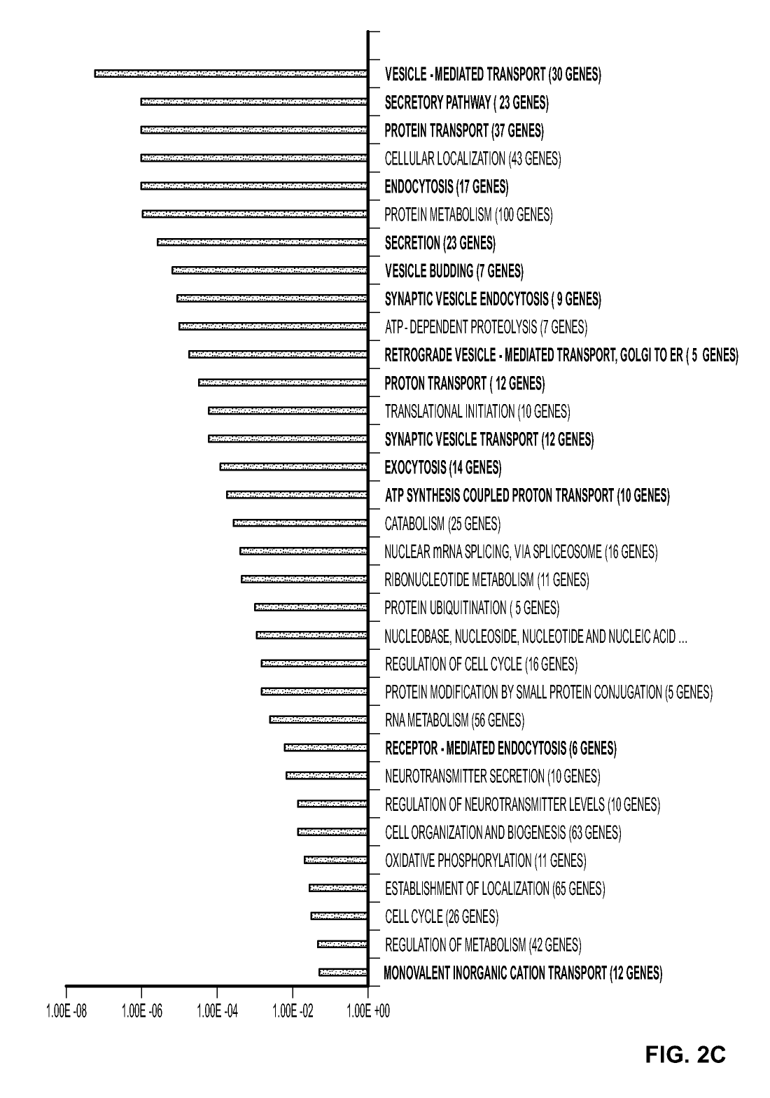

It was found that Sindbis productively infects Drosophila cells (FIG. 1A) and that this infection was dependent upon endosomal acidification for infection because treatment with ammonium chloride inhibited infection (FIG. 1B). Next, a high-content assay was optimized in a 384-well format using dsRNAs against luciferase as a negative control, dsRNA against GFP and the vATPase component vha26 (required for endosomal acidification) as viral and cellular positive controls, respectively (FIG. 1C). For screening, cells were seeded onto pre-arrayed 384 well plates, incubated for 3 days and infected with Sindbis for 36 hours. The genome-wide screen was performed in duplicate and 318 genes were identified (2% of the Drosophila genome) that when silenced had an impact on the percentage of Sindbis virus infected cells with a robust Z score of >2 or <2 in both replicates (P<0.05). (FIG. 2A). The analysis revealed that the data set was enriched for genes that have homologues in both humans and mosquitoes (p<0.001). Moreover, Drosophila-specific genes were greatly under-represented (p<0.001) (FIG. 2B). This demonstrates that most genes identified in the screen were conserved in the natural hosts of Sindbis. Bioinformatics was used to identify enriched Gene Ontology Biological Process categories and included many involved in intracellular vesicular trafficking (FIG. 2C, red).

### Example 2

### Characterization of Genes Identified in the Screen

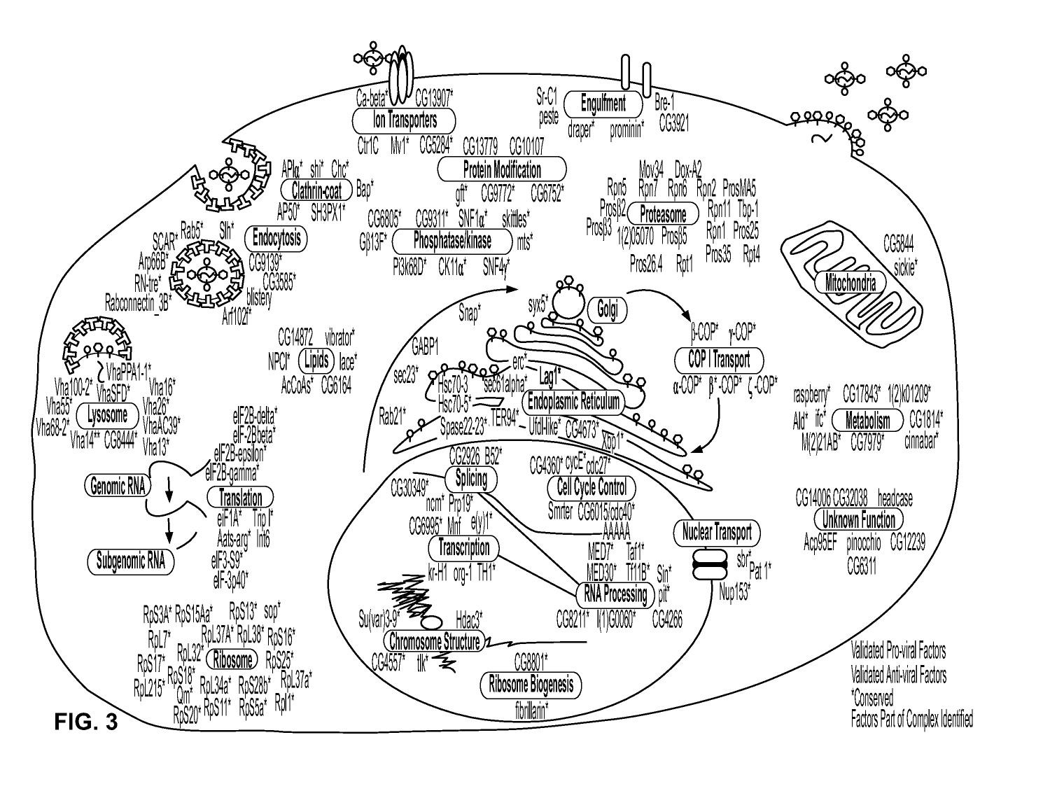

Of the 318 genes identified in the screen, 114 were cytotoxic (Robust Z-score<−2 in duplicate wells; ˜30% decrease in cell number). For validation, independent dsRNAs were generated against 231 of the 318 genes based upon conservation with relevant hosts (mosquitoes and humans). This gene library consisted of 149 genes which were non-toxic when silenced, 31 genes that had an effect on viability but had conserved protein domains, and 51 genes that were comprised of a representative subunit of well-characterized complexes (e.g. proteasome). The remaining genes were cytotoxic and not studied further. Of the 231 genes examined in greater detail, 178 validated (75%) of which 129 genes (73%) promoted Sindbis infection and 49 genes (27%) restricted infection (Table 1 below). Through functional annotation; the genes were placed into cellular pathways, subcellular compartments, and protein complexes demonstrating their role in viral infection (FIG. 3). Multiple pathways and complexes known to be involved in Sindbis replication were identified, further validating the gene-set (e.g., 11/13 components of the vATPase).

In particular, a large number of candidate genes were likely involved in viral entry, including genes involved in clathrin-mediated endocytosis (16 genes), vacuolar acidification (11/13 known components) and lipid biogenesis (6 genes) (FIG. 2C, FIG. 3). Previous studies have established an entry requirement for an as yet unidentified protein receptor for Sindbis, and plasma membrane-associated transporters are a common class of viral cellular receptors.

TABLE 1
Results of Drosophila Whole Genome RNAi Screen

| | Drosophila Whole Genome RNAi Screen | | | | |
|---|---|---|---|---|---|
| | Primary screen | Primary screen | Secondary Screen | Secondary Screen | |
| | Average GFP | Average Nuclei | Average GFP | Average Nuclei | |
| Gene Name | Z-score | Z-score | Z-score | Z-score | Category |
| Cdc27 | −13.6 | −0.6 | −3.3 | −3.1 | cell cycle |
| | | | | | control |
| CG4360 | 2.9 | −3.6 | 2.6 | 0.1 | cell cycle |
| | | | | | control |
| CG6015 | 3.0 | −3.7 | 1.7 | −4.8 | cell cycle |
| | | | | | control |
| CycE | 3.6 | −7.2 | 3.4 | −1.5 | cell cycle |
| | | | | | control |
| smrter | −5.7 | −1.1 | −1.7 | −1.8 | cell cycle |
| | | | | | control |
| CG4557 | −5.0 | −0.5 | −1.7 | −0.5 | chromosome |
| | | | | | structures |
| Hdac3 | −3.0 | −1.5 | −1.8 | −1.7 | chromosome |
| | | | | | structures |
| Su(var)3-9 | −5.4 | −4.7 | −1.5 | −2.3 | chromosome |
| | | | | | structures |
| tlk | 2.8 | −2.0 | 3.0 | −1.9 | chromosome |
| | | | | | structures |
| alpha-Adaptin | −9.1 | 0.2 | −2.3 | −0.7 | clathrin-coat |
| Ap 1alpha | −5.7 | −1.1 | −1.6 | −0.8 | clathrin-coat |
| AP-50 | −5.4 | 0.3 | −3.5 | −0.5 | clathrin-coat |
| Bap | −9.1 | −2.0 | −2.7 | −0.4 | clathrin-coat |
| CG9139 | −2.38 | 0.76 | −1.3 | −2.0 | clathrin-coat |
| Chc | −14.6 | −0.5 | −4.0 | −0.7 | clathrin-coat |
| SH3PX1 | −2.2 | −0.2 | −2.4 | −0.1 | clathrin-coat |
| shi | −16.8 | −2.5 | −4.2 | −0.9 | clathrin-coat |
| alphaCop | −16.8 | −4.0 | −4.3 | −3.9 | cop I |
| | | | | | transport |
| betaCop | −16.5 | −5.1 | −4.9 | −3.0 | cop I |
| | | | | | transport |
| beta′Cop | −15.5 | −4.4 | −4.8 | −4.4 | cop I |
| | | | | | transport |
| gammaCop | −17.7 | −5.0 | −5.1 | −3.7 | cop I |
| | | | | | transport |
| zetaCOP | −11.3 | −5.7 | −4.5 | −3.4 | cop I |
| | | | | | transport |
| Arf102F | −4.1 | −3.3 | −2.1 | −0.2 | cytoskeleton |
| Arp66B | −3.9 | −0.8 | −4.4 | 0.0 | cytoskeleton |
| blistery | −3.2 | −0.4 | −1.6 | 1.0 | cytoskeleton |
| CG3960 | −3.5 | 0.7 | −1.7 | 0.4 | cytoskeleton |
| Hem | −4.1 | −0.3 | −1.8 | 0.1 | cytoskeleton |
| SCAR | −3.2 | 0.1 | −2.0 | −0.6 | cytoskeleton |
| CecC | 2.4 | −1.8 | 1.6 | −0.4 | defense |
| | | | | | response |
| hdc | 2.3 | −1.4 | 3.9 | −0.3 | defense |
| | | | | | response |
| CG4673 | −3.1 | −1.0 | −2.7 | −1.4 | endoplasmic |
| | | | | | reticulum |
| CG9920 | −2.5 | 0.7 | −2.1 | −0.4 | endoplasmic |
| | | | | | reticulum |
| Hsc70-3 | −5.4 | −9.0 | −2.1 | −1.1 | endoplasmic |
| | | | | | reticulum |
| Hsc70-5 | 3.4 | −5.4 | 2.5 | −0.3 | endoplasmic |
| | | | | | reticulum |
| Lag1 | −10.7 | −0.8 | −3.1 | 0.0 | endoplasmic |
| | | | | | reticulum |
| Sec61alpha | −4.2 | −1.2 | −1.8 | −0.1 | endoplasmic |
| | | | | | reticulum |
| Spase22-23 | −3.2 | −1.1 | −1.8 | −0.2 | endoplasmic |
| | | | | | reticulum |
| TER94 | −12.6 | −5.7 | −4.8 | −3.0 | endoplasmic |
| | | | | | reticulum |
| Ufd1-like | −10.3 | −1.3 | −1.5 | −1.5 | endoplasmic |
| | | | | | reticulum |
| Xbp1 | −2.8 | 0.4 | −4.4 | −9.1 | endoplasmic |
| | | | | | reticulum |
| CG3585 | −4.8 | 0.6 | −3.9 | 0.3 | endosome |
| Rab5 | −14.7 | −6.3 | −5.1 | −2.1 | endosome |
| Rabconnectin 3B | −2.7 | −0.3 | −4.0 | 0.2 | endosome |
| RN-tre | −4.2 | −3.0 | −4.3 | −0.6 | endosome |
| SIh | −9.0 | −1.3 | −4.0 | −0.6 | endosome |
| Bre-1 | −4.5 | −1.1 | −2.6 | 0.3 | engulfment |
| CG6479 | 3.4 | −1.1 | 6.9 | −0.9 | engulfment |
| draper | −9.9 | −2.3 | −1.5 | 1.4 | engulfment |
| peste | −5.5 | 0.9 | −3.6 | −0.1 | engulfment |
| Prominin | −4.4 | −0.2 | −2.0 | 0.1 | engulfment |
| Sr-CI | 2.0 | −0.8 | 5.0 | −0.3 | engulfment |
| Cpr62Ba | −3.7 | 0.7 | −3.1 | 1.0 | insect |
| | | | | | development |
| Cpr97Eb | −7.7 | 0.8 | −1.5 | 0.8 | insect |
| | | | | | development |
| Patsas | −2.2 | −0.2 | −1.8 | −0.1 | insect |
| | | | | | development |
| pinocchio | −2.8 | −0.2 | −2.2 | −0.7 | insect |
| | | | | | development |
| Ca-beta | −2.7 | 0.1 | −1.9 | 0.3 | ion |
| | | | | | transporters |
| CG13907 | −3.2 | −0.4 | −2.1 | 0.4 | ion |
| | | | | | transporters |
| CG5284 | −13.3 | 0.0 | −4.7 | −0.5 | ion |
| | | | | | transporters |
| Ctr1C | −3.3 | 0.7 | −3.1 | 0.0 | ion |
| | | | | | transporters |
| Mvl | −3.0 | −0.3 | −3.7 | 0.1 | ion |
| | | | | | transporters |
| Orct2 | −6.2 | 0.2 | −2.0 | −0.6 | ion |
| | | | | | transporters |
| AcCoAS | −5.2 | −2.5 | −4.1 | −1.4 | lipids |
| CG14872 | −2.6 | 0.6 | −2.2 | −0.8 | lipids |
| CG6164 | −14.0 | −4.0 | −2.8 | 0.4 | lipids |
| lace | −13.2 | 1.2 | −3.5 | −0.4 | lipids |
| NPC1 | −2.4 | 0.2 | −1.5 | −0.3 | lipids |
| vib | −2.7 | −0.7 | −3.6 | −0.5 | lipids |
| CG8444 | −2.9 | 0.0 | −3.3 | −0.4 | lysosome |
| Vha100-2 | −4.3 | −0.8 | not tested | not tested | lysosome |
| Vha13 | −2.4 | −0.4 | not tested | not tested | lysosome |
| Vha14 | −4.8 | −0.8 | not tested | not tested | lysosome |
| Vha16 | −6.7 | −0.6 | not tested | not tested | lysosome |
| Vha26 | −6.2 | −0.5 | −3.9 | 0.0 | lysosome |
| Vha55 | −5.3 | −1.1 | not tested | not tested | lysosome |
| Vha68-2 | −2.1 | 0.2 | not tested | not tested | lysosome |
| VhaAC39 | −11.1 | −0.7 | not tested | not tested | lysosome |
| VhaPPA1-1 | −14.5 | 0.2 | not tested | not tested | lysosome |
| VhaSFD | −6.6 | −0.3 | −4.3 | 0.3 | lysosome |
| Ald | −6.6 | −3.5 | −6.5 | −0.2 | metabolism |
| CG17843 | −2.0 | 1.3 | −1.6 | 0.2 | metabolism |
| CG1814 | −2.4 | 0.1 | −5.2 | −0.2 | metabolism |
| CG31198 | −3.0 | 1.3 | −1.7 | −0.4 | metabolism |
| CG3621 | −1.9 | −0.6 | −1.5 | 1.3 | metabolism |
| CG5537 | −4.1 | 0.1 | −3.8 | 0.2 | metabolism |
| CG7979 | −7.8 | 0.0 | −2.2 | −0.5 | metabolism |
| cinnabar | 2.2 | −1.7 | 6.4 | −0.8 | metabolism |
| Esterase P | −5.9 | −2.5 | −1.7 | 0.0 | metabolism |
| ifc | −10.2 | −0.6 | −8.1 | 0.2 | metabolism |
| l(2)k01209 | −3.1 | 0.6 | −2.4 | 0.0 | metabolism |
| M(2)21AB | −18.3 | −4.5 | −7.3 | −3.5 | metabolism |
| raspberry | −7.4 | −3.1 | −2.4 | −1.2 | metabolism |
| CG1746 | 3.3 | −3.5 | 2.8 | −1.8 | mitochondria |
| CG40451 | 9.6 | −1.0 | 3.3 | −0.9 | mitochondria |
| CG5844 | −7.0 | −3.3 | −8.3 | −6.1 | mitochondria |
| Nos | −2.2 | 0.0 | −2.7 | −0.4 | mitochondria |
| sickie | 2.2 | −1.9 | 2.2 | 0.1 | mitochondria |
| Nup153 | −6.2 | −8.1 | −2.9 | −1.5 | nuclear |
| | | | | | transport |
| Nup98 | −4.6 | −6.7 | −3.0 | −1.4 | nuclear |
| | | | | | transport |
| Pat1 | −3.5 | 0.7 | −2.0 | 0.0 | nuclear |
| | | | | | transport |
| sbr | 2.7 | −2.4 | 4.3 | −3.4 | nuclear |
| | | | | | transport |
| CG6805 | −3.2 | −1.0 | −1.5 | 0.7 | phosphatase/ |
| | | | | | kinase |
| CG9311 | −3.9 | 0.1 | −2.8 | 1.3 | phosphatase/ |
| | | | | | kinase |
| CkIIalpha | 2.2 | −1.3 | 6.5 | 0.0 | phosphatase/ |
| | | | | | kinase |
| Gbeta13F | −2.1 | 0.7 | −2.3 | 0.1 | phosphatase/ |
| | | | | | kinase |
| mts | −7.8 | −1.7 | −1.7 | −4.4 | phosphatase/ |
| | | | | | kinase |
| Pi3K68D | −8.6 | −2.1 | −3.1 | 0.4 | phosphatase/ |
| | | | | | kinase |
| skittles | −5.0 | −0.1 | −3.6 | −0.5 | phosphatase/ |
| | | | | | kinase |
| SNF1a | −3.5 | −1.5 | −3.5 | −1.5 | phosphatase/ |
| | | | | | kinase |
| SNF4Ag | −2.2 | 0.6 | −3.7 | −2.2 | phosphatase/ |
| | | | | | kinase |
| Dox-A2 | −18.9 | −8.2 | not tested | not tested | proteasome |
| l(2)05070 | −13.0 | −5.5 | not tested | not tested | proteasome |
| Move34 | −9.3 | −4.9 | not tested | not tested | proteasome |
| Pros25 | −8.4 | −6.2 | not tested | not tested | proteasome |
| Pros26.4 | −11.5 | −5.2 | not tested | not tested | proteasome |
| Pros35 | −12.1 | −4.6 | not tested | not tested | proteasome |
| Prosbeta2 | −3.6 | −4.0 | not tested | not tested | proteasome |
| Prosbeta3 | −3.7 | −4.3 | not tested | not tested | proteasome |
| Prosbeta5 | −5.7 | −7.4 | not tested | not tested | proteasome |
| ProsMA5 | −2.8 | −7.1 | not tested | not tested | proteasome |
| Rpn1 | −6.0 | −5.7 | not tested | not tested | proteasome |
| Rpn11 | −13.9 | −5.4 | not tested | not tested | proteasome |
| Rpn2 | −12.3 | −9.5 | not tested | not tested | proteasome |
| Rpn5 | −4.5 | −3.5 | not tested | not tested | proteasome |
| Rpn6 | −23.9 | −7.5 | −10.8 | −29.9 | proteasome |
| Rpn7 | −13.2 | −5.8 | not tested | not tested | proteasome |
| Rpt1 | −6.3 | −4.9 | not tested | not tested | proteasome |
| Rpt4 | −11.2 | −5.2 | not tested | not tested | proteasome |
| Tbp-1 | −18.4 | −9.0 | not tested | not tested | proteasome |
| CG10107 | −4.8 | −1.7 | −2.3 | −1.2 | protein |
| | | | | | modification |
| CG13779 | −2.2 | −2.2 | −2.8 | −1.2 | protein |
| | | | | | modification |
| CG17572 | −2.4 | −0.7 | −2.5 | 0.1 | protein |
| | | | | | modification |
| CG5976 | −2.2 | 0.2 | −2.3 | 0.2 | protein |
| | | | | | modification |
| CG6752 | 11.4 | −5.5 | 1.8 | −0.3 | protein |
| | | | | | modification |
| CG9772 | −2.5 | 1.0 | −2.6 | 0.1 | protein |
| | | | | | modification |
| CG9934 | −3.8 | −1.1 | −2.7 | −0.1 | protein |
| | | | | | modification |
| effete | −4.7 | −2.4 | −2.5 | 0.0 | protein |
| | | | | | modification |
| gft | −5.7 | −0.5 | −3.9 | 0.3 | protein |
| | | | | | modification |
| Roc1a | −2.9 | −2.5 | −2.7 | −2.2 | protein |
| | | | | | modification |
| Qm | −2.2 | −4.6 | not tested | not tested | ribosome |
| RpI1 | 2.8 | −1.3 | not tested | not tested | ribosome |
| RpII215 | −3.3 | −8.1 | not tested | not tested | ribosome |
| RpL32 | −2.6 | −5.9 | not tested | not tested | ribosome |
| RpL34a | 2.4 | −7.3 | not tested | not tested | ribosome |
| RpL37A | −5.3 | −5.7 | not tested | not tested | ribosome |
| RpL38 | −6.9 | −5.1 | not tested | not tested | ribosome |
| RpL7 | 2.2 | −7.7 | not tested | not tested | ribosome |
| RpS13 | 3.1 | −3.9 | 13.2 | −4.0 | ribosome |
| RpS15Aa | −3.3 | −5.1 | not tested | not tested | ribosome |
| RpS16 | −0.6 | −7.5 | not tested | not tested | ribosome |
| RpS17 | 3.2 | −3.6 | not tested | not tested | ribosome |
| RpS18 | −5.1 | −4.7 | not tested | not tested | ribosome |
| RpS20 | 2.9 | −1.9 | not tested | not tested | ribosome |
| RpS25 | 3.5 | −1.2 | not tested | not tested | ribosome |
| RpS28b | 3.1 | −6.7 | not tested | not tested | ribosome |
| RpS3A | 4.5 | −6.8 | not tested | not tested | ribosome |
| RpS5a | 4.8 | −6.0 | not tested | not tested | ribosome |
| sop | −2.6 | −4.8 | not tested | not tested | ribosome |
| CG8801 | 2.3 | −1.2 | 1.9 | −0.1 | ribosome |
| | | | | | biogenesis |
| fibrillarin | 2.1 | −0.4 | 9.4 | −0.4 | ribosome |
| | | | | | biogenesis |
| CG4266 | −4.9 | −1.5 | −4.4 | −0.3 | RNA |
| | | | | | processing |
| CG8211 | −3.5 | −1.4 | −3.0 | −1.5 | RNA |
| | | | | | processing |
| l(1)00060 | −6.5 | −0.9 | −2.4 | −1.3 | RNA |
| | | | | | processing |
| MED30 | 2.3 | −0.1 | 10.5 | −0.7 | RNA |
| | | | | | processing |
| MED7 | 3.3 | −3.0 | not tested | not tested | RNA |
| | | | | | processing |
| Pit | 6.6 | 1.2 | 4.2 | −0.7 | RNA |
| | | | | | processing |
| Sin | 3.7 | −2.3 | 4.7 | −1.4 | RNA |
| | | | | | processing |
| Taf1 | 2.7 | −1.5 | 13.2 | −1.7 | RNA |
| | | | | | processing |
| TfIIB | 2.9 | −2.2 | 13.4 | −3.1 | RNA |
| | | | | | processing |
| B52 | 1.9 | −1.7 | 9.4 | −2.0 | splicing |
| CG2926 | −6.5 | 0.9 | −2.2 | −0.7 | splicing |
| CG30349 | 2.6 | −1.1 | 7.7 | 0.0 | splicing |
| ncm | −4.4 | −6.7 | −3.6 | −7.2 | splicing |
| peanuts | −2.4 | −2.8 | −2.5 | −1.6 | splicing |
| Prp19 | −3.5 | −4.9 | −2.2 | −1.3 | splicing |
| CG6995 | −7.6 | 0.4 | −3.1 | −0.4 | transcription |
| crc | −2.7 | 0.7 | −1.5 | 0.7 | transcription |
| e(y)1 | 3.3 | −1.0 | 10.5 | −1.2 | transcription |
| Kr-h1 | −2.1 | 1.1 | −1.6 | 0.5 | transcription |
| Mnf | 2.2 | −1.9 | 7.8 | 0.1 | transcription |
| org-1 | 2.2 | −2.0 | 1.6 | −0.5 | transcription |
| TH1 | 3.9 | −2.0 | 8.7 | −0.4 | transcription |
| Aats-arg | 2.5 | −2.1 | 5.5 | −1.7 | translation |
| eiF1A | 2.9 | −2.0 | 3.1 | −2.6 | translation |
| eIF2B-beta | 2.5 | −1.2 | not tested | not tested | translation |
| eIF2B-delta | 2.8 | −1.6 | not tested | not tested | translation |
| eIF2B-epsilon | 3.3 | −3.2 | not tested | not tested | translation |
| eIF2B-gamma | 3.1 | −0.5 | 11.7 | −1.3 | translation |
| eIF-3p40 | 2.9 | −2.0 | 5.7 | −1.2 | translation |
| eIF3-S9 | 2.4 | −4.6 | 9.2 | −2.4 | translation |
| Int6 | 3.4 | −4.5 | not tested | not tested | translation |
| RpS11 | 5.0 | −6.8 | 2.3 | 0.3 | translation |
| Trip1 | 4.9 | −5.7 | 13.2 | −2.7 | translation |
| GABPI | −2.2 | 0.0 | −2.7 | −0.4 | transport |
| Rab21 | −9.8 | 0.9 | −3.4 | 0.1 | transport |
| sec23 | −4.7 | −4.6 | −5.1 | −2.4 | transport |
| Snap | −13.4 | −5.8 | −7.1 | −7.2 | transport |
| Syx5 | −10.9 | −2.7 | −4.2 | −2.7 | transport |
| Acp95EF | −2.2 | −1.0 | −7.0 | −1.3 | unknown |
| CG12239 | 2.2 | −0.7 | 4.0 | −1.0 | unknown |
| CG14006 | −2.4 | 1.1 | −1.7 | 0.3 | unknown |
| CG14894 | −2.6 | −0.4 | −3.4 | 0.7 | unknown |
| CG32038 | −3.9 | −0.5 | −4.9 | 0.0 | unknown |

### Example 3

### dNRAMP Mediates Viral Entry

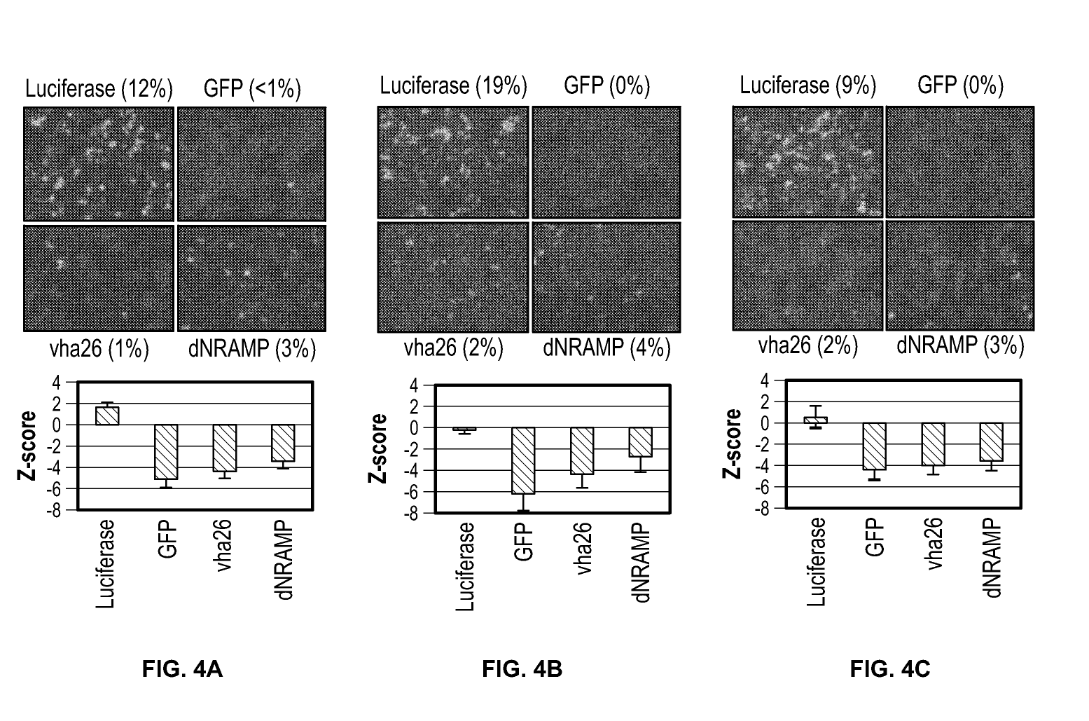

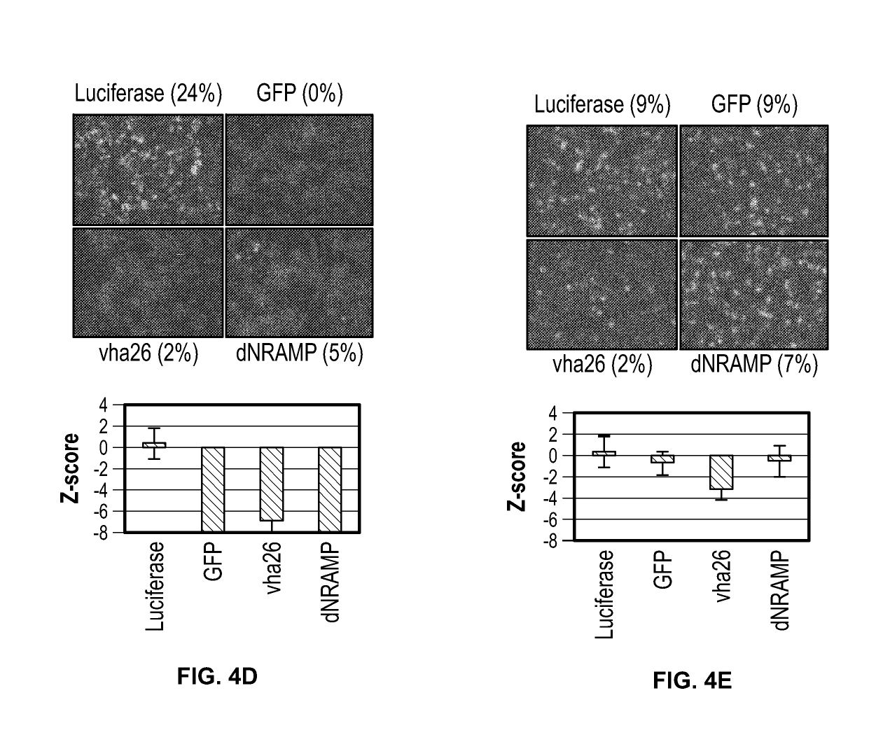

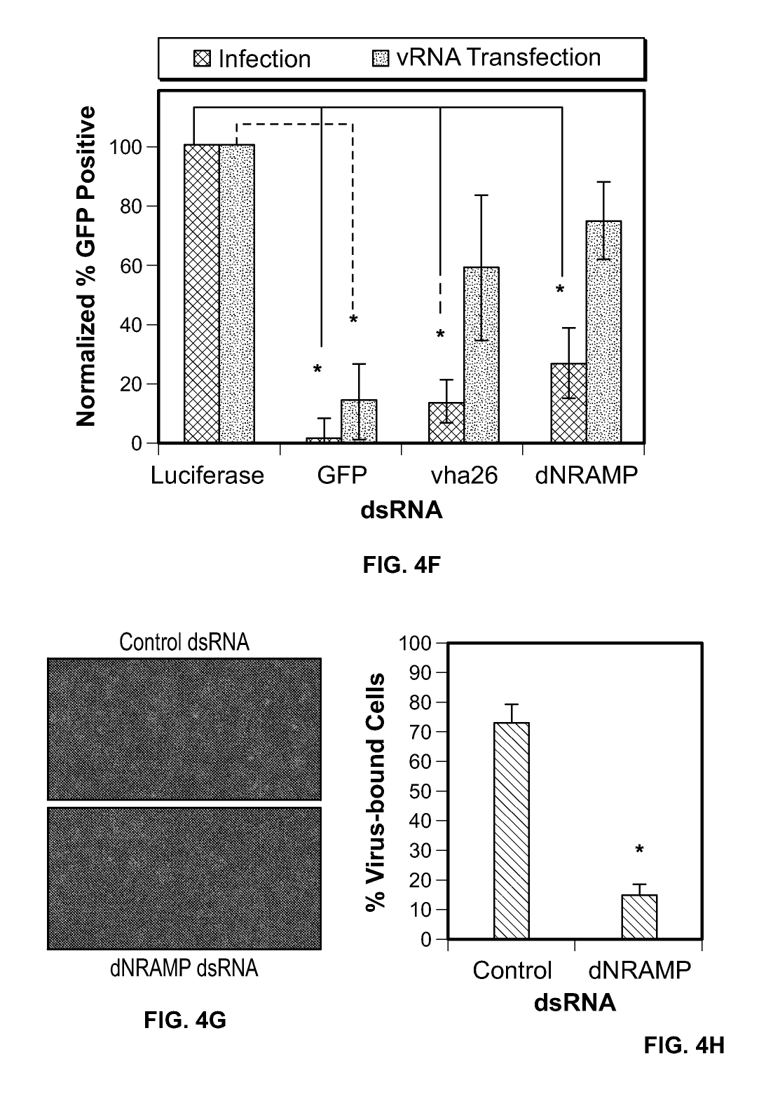

To characterize the requirement for dNRAMP during Sindbis infection, independent dsRNAs were tested in both the original cell line used for screening (FIG. 4A) as well as Kc167, another Drosophila cell line (FIG. 4B), and it was found that Sindbis infection was significantly decreased in both cases compared to negative controls (luciferase dsRNA).

Cells were treated with dsRNA against GFP and vha26 (required for endosomal acidification) as positive controls against viral or cellular genes, respectively (FIGS. 4A-D). In addition another more pathogenic Sindbis strain, dsTE12H, was also dependent on dNRAMP for infection (FIG. 4C). Since Sindbis virus strains HRsp and dsTE12H are lab-adapted, a wild type strain, S.A.AR86, was also tested. Like Sindbis virus, West Nile virus is an arbovirus (of the Flavivirus family) which is known to enter cells via clathrin-mediated endocytosis and traffics to an acidified compartment for fusion. It was found that while many of the canonical components of the clathin-mediated endocytic pathway and the vATPase acidification machinery were required for both Sindbis and West Nile virus infection of Drosophila cells, dNRAMP was completely dispensable for West Nile virus infection (FIG. 4D). In addition, it was found that dNRAMP was dispensible for infection with Vesicular Stomatitis Virus and Drosophila C virus two viruses that are dependent upon clathrin-mediated endocytosis for entry. These data show that dNRAMP was not ubiquitously involved in the clathrin-dependent uptake of viruses in general but rather specifically required for Sindbis infection.

The step in the Sindbis virus lifecycle that is dependent upon dNRAMP was then investigated and it was found that silencing attenuated both viral antigen production, as measured by immunoblot, and viral RNA replication, as determined by RT-PCR, thereby suggesting a role at or upstream of RNA replication. To determine if the requirement for dNRAMP was at the level of entry, a viral RNA-bypass assay was performed. When transfected into cells, genomic viral RNA can bypass any entry requirements and initiate replication launching an infection. Therefore, the production of virally-expressed GFP was compared between infection (entry-dependent) and viral RNA transfection (entry-independent) in cells depleted for the control luciferase to the vATPase or dNRAMP. Using this assay, it was found that the requirement for dNRAMP, as well as the vATPase, was largely bypassed by viral RNA transfection suggesting a role for dNRAMP in entry (FIG. 4F). In contrast, treatment of cells with dsRNA against the virally expressed reporter GFP could not be bypassed.

Since dNRAMP is a plasma membrane-localized transporter, the requirement of dNRAMP for virus binding to cells was assayed. For these experiments, cells were pre-treated with dsRNA targeting dNRAMP or the negative control luciferase, and then challenged with biotinylated infectious Sindbis virus at 4° C. Free virus was removed, and bound virus was visualized with Streptavidin-Texas Red. Cells (FIG. 4G). Quantification revealed that cells depleted of dNRAMP showed a ˜5-fold reduction in binding compared to control dsRNA treated cells (FIG. 4H).

To characterize the requirement for dNRAMP during Sindbis virus infection, we tested independent dsRNAs in both the original Drosophila cell line used for screening (FIG. 11A) as well as Kc167, another Drosophila cell line (FIG. 11B), and found that infection with Sindbis virus HRsp expressing a GFP reporter gene was significantly decreased in both cases compared to negative controls (luciferase dsRNA). We treated cells with dsRNA against GFP and vha26 (required for endosomal acidification) as positive controls against viral or cellular genes, respectively (FIGS. 11A-D). In addition, we found that another, more pathogenic Sindbis virus strain, dsTE12H, was also dependent on dNRAMP for infection (FIG. 11C). Since Sindbis virus strains HRsp and dsTE12H are lab-adapted, we also tested whether the wild type strain S.A.AR86 was also dependent upon dNRAMP for infection. Indeed, we found that this strain was also dependent upon dNRAMP (FIG. 11D). The Sindbis S.A.AR86 strain was a replicon which is unable to spread demonstrating that the requirement is during the first round of infection. Like Sindbis virus, West Nile Virus is an arbovirus (of the Flavivirus family) which is known to enter cells via clathrin-mediated endocytosis and traffics to an acidified compartment for fusion. Using wild type West Nile virus we found that while many of the canonical components of the clathrin-mediated endocytic pathway and the vATPase acidification machinery were required for both Sindbis virus and West Nile Virus infection of Drosophila cells, dNRAMP was completely dispensable for West Nile virus infection (FIG. 11E). In addition, we found that dNRAMP was dispensable for infection with Vesicular Stomatitis Virus (VSV) an additional virus that is dependent upon clathrin-mediated endocytosis for entry (FIG. 11F). These data suggest that dNRAMP was not ubiquitously involved in the clathrin-dependent uptake of viruses in general but rather specifically required for Sindbis virus infection.

### Example 4

### dNRAMP is Required for Sindbis Infection of Adult Flies

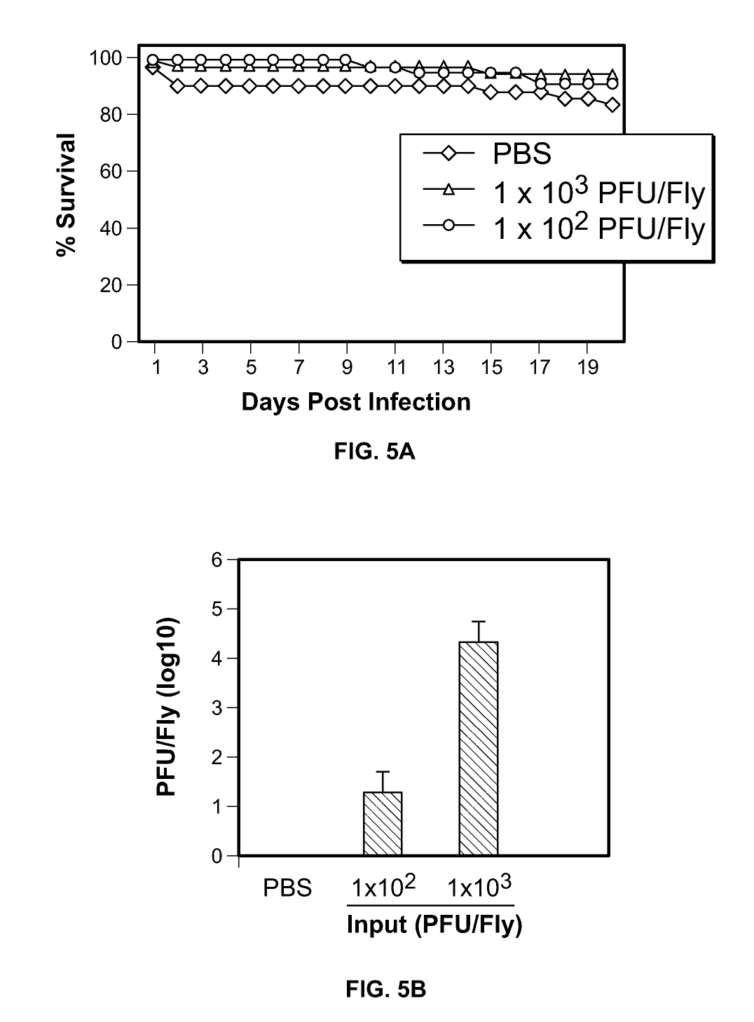

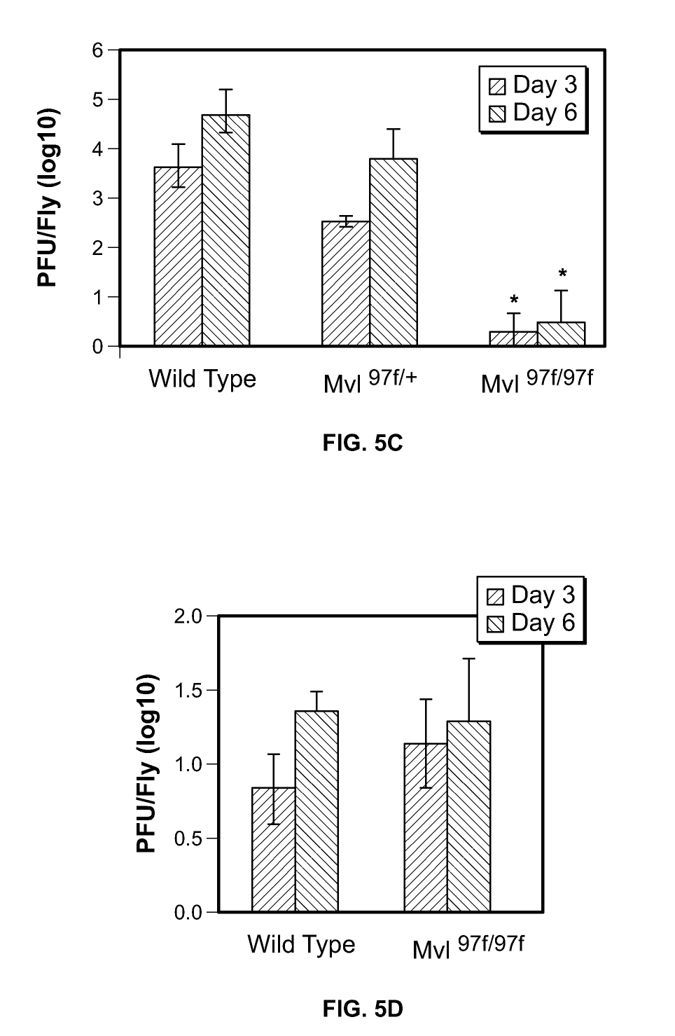

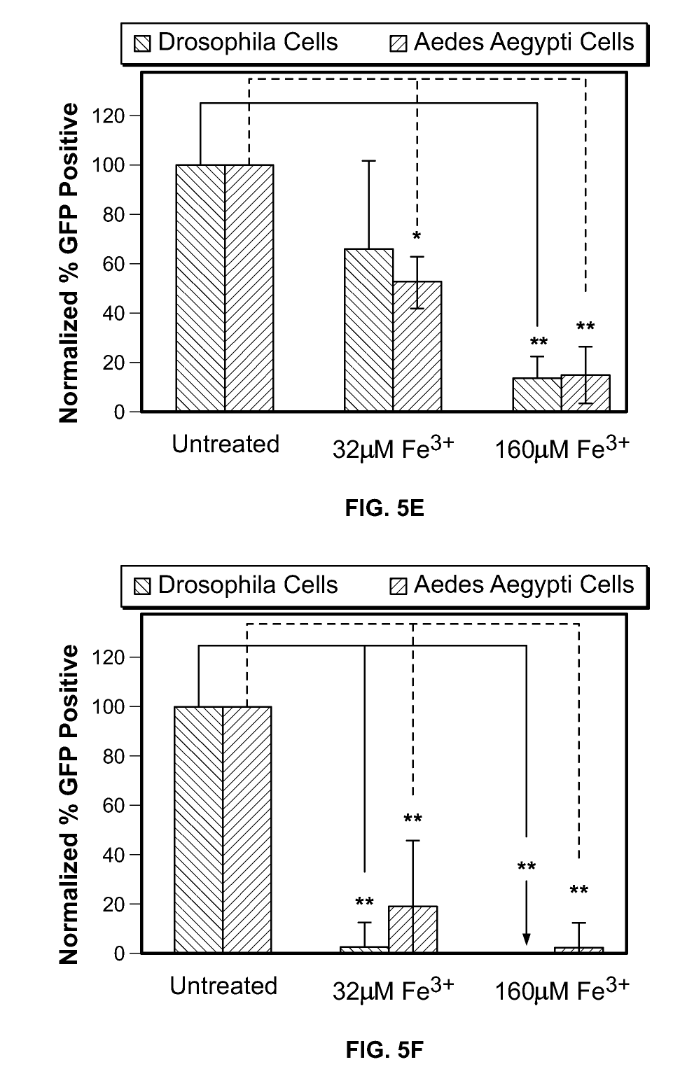

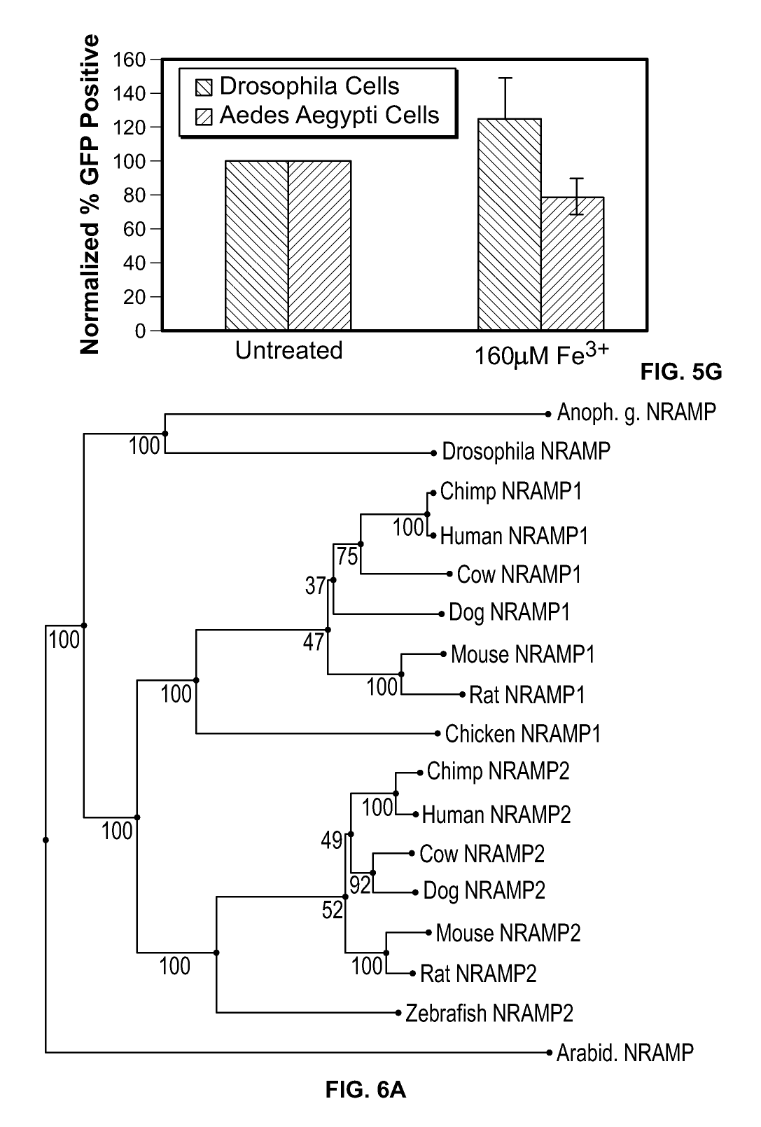

Whether dNRAMP is required for infection not only of tissue culture cells, but also of adult flies was determined. Sindbis virus infection of both the natural mosquito host and adult flies is non-lethal (FIG. 5A) and the generation of infectious virus in vivo was dose-dependent (FIG. 5B). A transposon insertion allele of dNRAMP (Mvl^{97f}) has been previously characterized as strong loss-of-function allele that behaves as a null allele in some behavioral assays. There was a 4-log reduction in viral titers of flies that were infected with Sindbis virus but deficient for dNRAMP (Mvl^{97f}) compared to heterozygous or wild-type controls while VSV infection was unaffected (FIGS. 5C and D, respectively). Altogether, these data illustrate that dNRAMP is required for infection both at the cellular and organismal level.

### Example 5

### Post-Translational Regulation of NRAMP by Iron Inhibits Sindbis Virus Infection in Insects

Since Drosophila is not the natural host of Sindbis virus, it was determined if NRAMP was also required for Sindbis virus infection of one of the natural mosquito hosts, Aedes aegypti. For these studies advantage was taken of the fact that NRAMP is a metal ion transporter sensitive to intracellular iron concentrations whereby high concentrations of iron lead to a reduction in NRAMP expression. NRAMP downregulation can occur on two levels. First, NRAMP2 is regulated through effects on mRNA stability; when iron levels are high NRAMP2 mRNA is degraded. Second, NRAMP is post-transcriptionally degraded; in the presence of high iron NRAMP interacts with recycling adaptor complexes and is removed from the cell surface for degradation. These mechanisms of regulation synergistically deplete NRAMP2 from cells. If NRAMP is a Sindbis virus receptor, high iron treatment would be predicted to attenuate infection. Indeed, iron treatment of Drosophila cells attenuated infection with both lab-adapted (FIG. 5E) and wild type (FIG. 5F) Sindbis virus while having no effect on infection with VSV (FIG. 5G). Likewise, treatment of Aedes Aegypti Aag-2 cells with iron also significantly impeded Sindbis virus infection of lab-adapted (HRsp) and wild type (S.A.AR86) Sindbis virus strains in a dose-dependent manner but not VSV (FIGS. 5E-G). Again, the use of the S.A.AR86 replicon that cannot spread, demonstrates that this requirement is during the first round of infection. Taken altogether the data demonstrates that dNRAMP is required for Sindbis virus entry into insects including vector mosquitoes.

### Example 6

### Sindbis Infection Treatment with High Iron Causes a Decrease in Infection

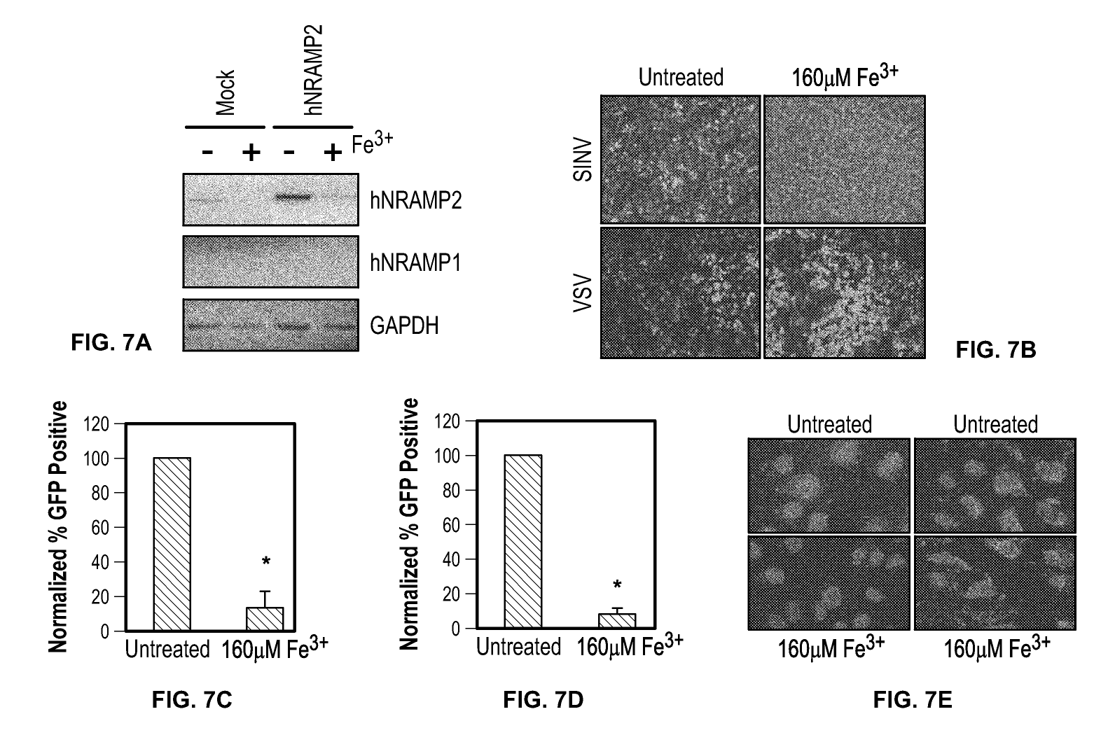

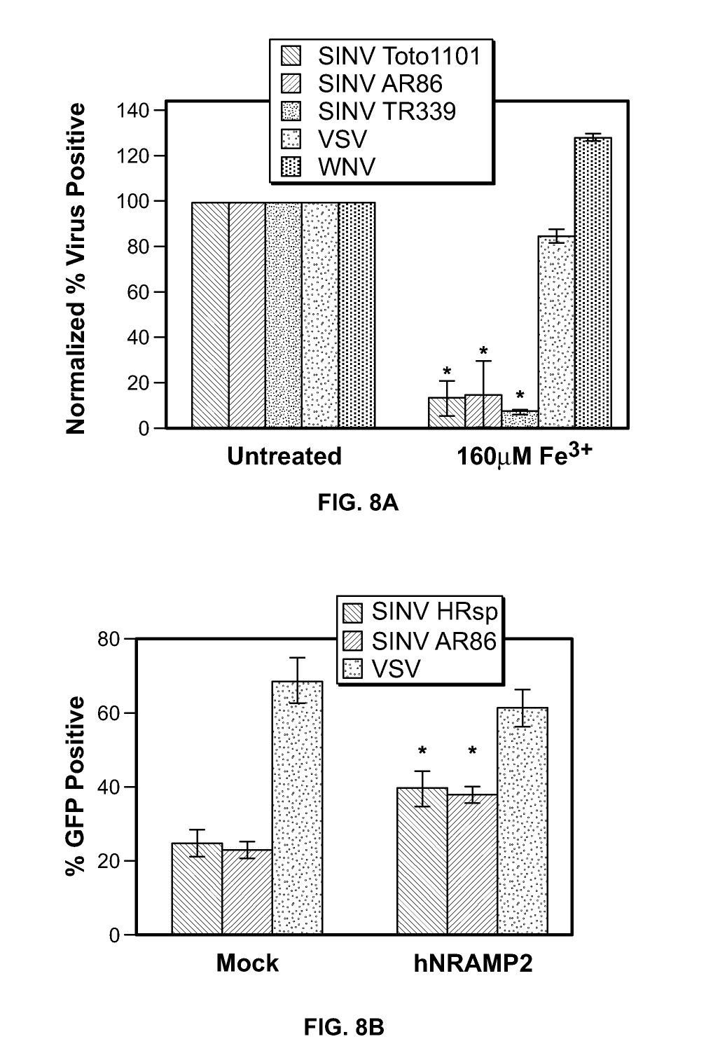

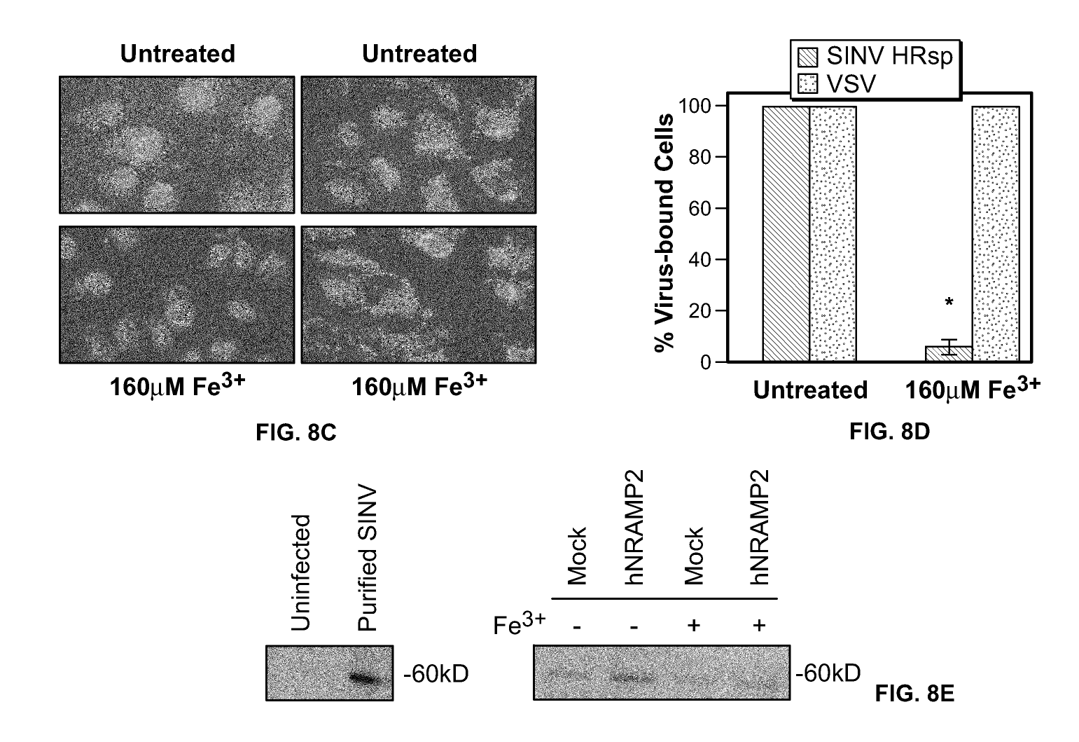

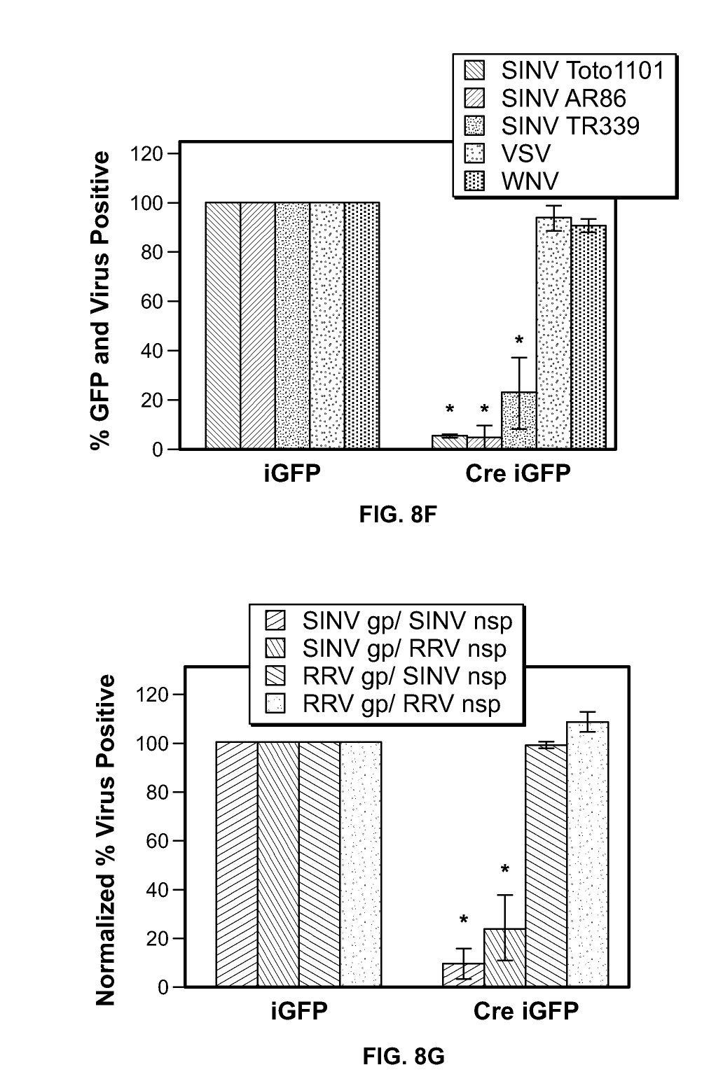

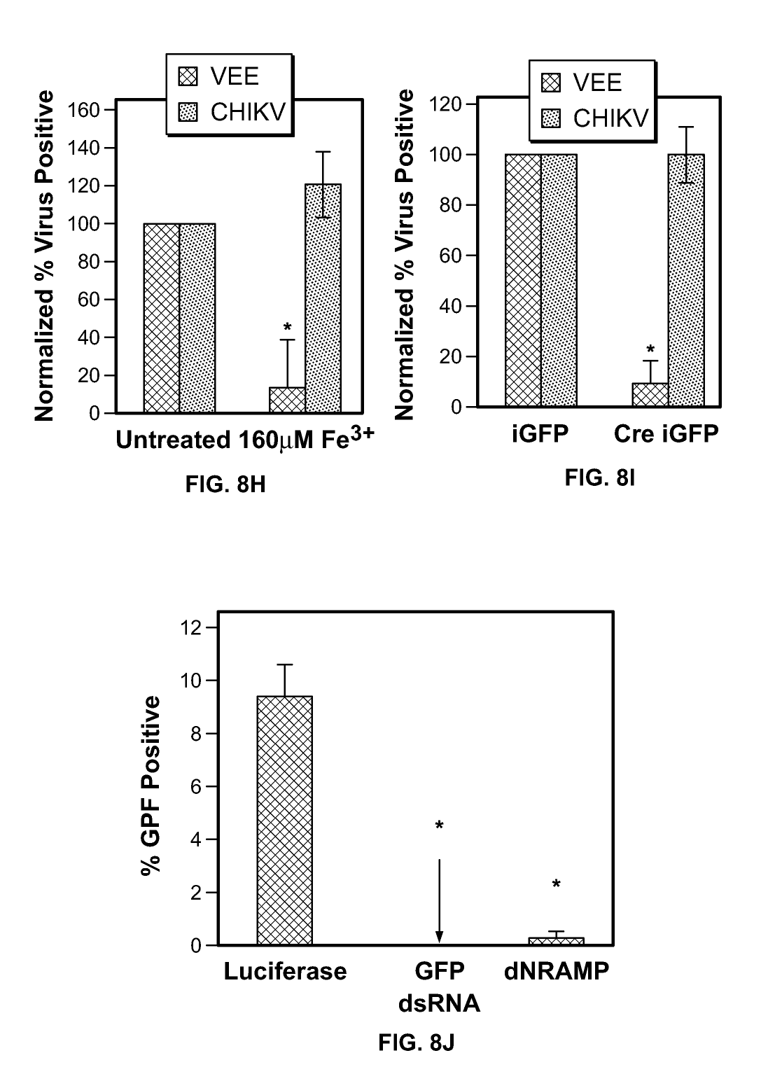

Due to the conservation between NRAMPs (FIGS. 6A-C), the role of hNRAMP in Sindbis infection of mammalian cells was assessed. hNRAMP1 has a restricted expression pattern, while NRAMP2 is more ubiquitously expressed. In addition, NRAMP1 is found predominantly in intracellular compartments, while NRAMP2 is expressed more ubiquitously at the plasma membrane and in recycling endosomes. Since Sindbis virus can infect many cell types and is thought to initially interact with its entry receptor(s) at the plasma membrane, NRAMP2 is more likely to function in entry. First, whether Sindbis virus infection is sensitive to iron as NRAMP2 expression is decreased by treatment with high iron was determined (FIG. 7A). Indeed, treatment of mammalian cells including human U2OS with iron significantly attenuated lab-adapted (Toto 1101) and wild type (S.A.AR86, TR339) Sindbis virus infection, and did so during the first round of infection because the two wild type strains are replicons unable to spread (FIG. 8A, FIG. 7B). Furthermore, iron treatment had no effect on West Nile virus or VSV infection (FIG. 8A) demonstrating a specific dependency for Sindbis virus. We also found that iron-treated human 293T cells and mouse embryonic fibroblasts (MEFs) were refractory to Sindbis virus infection (FIGS. 7C and 7D, respectively).

### Example 7

### NRAMP2 Facilitates Infection and Binding of Sindbis Virus in Mammalian Cells

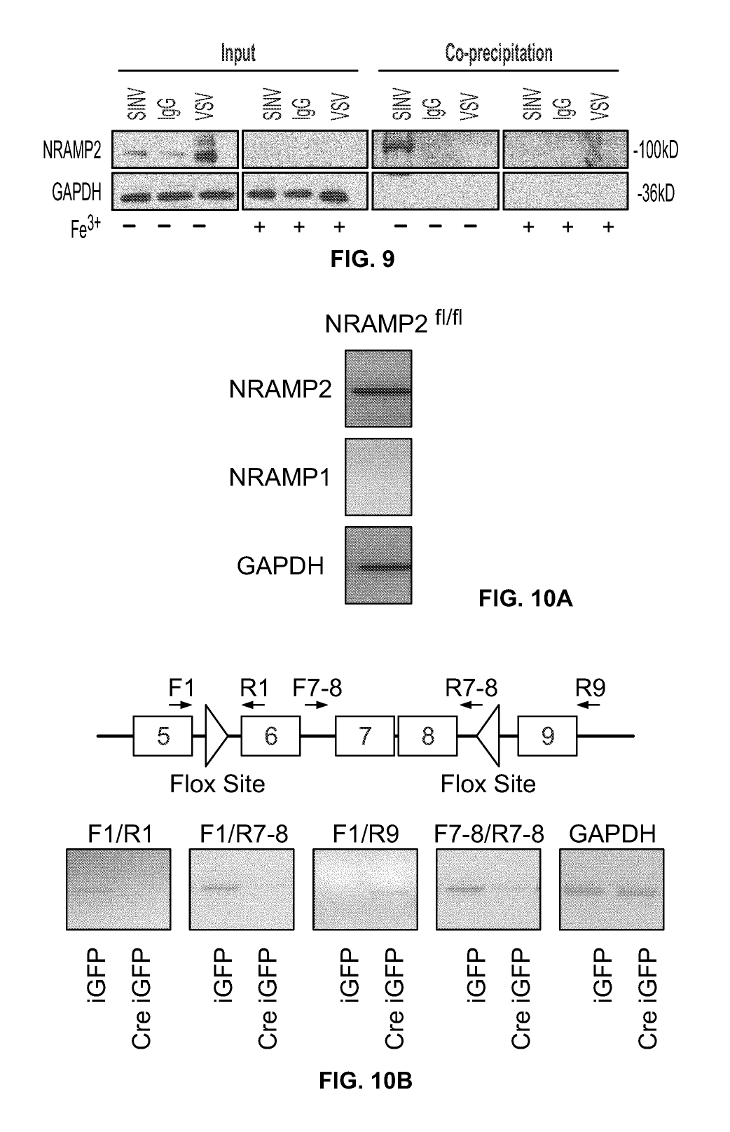

As many receptors are rate-limiting for viral infection, whether NRAMP2 over-expression impacted Sindbis virus infection was tested. Indeed, over-expression of NRAMP2 led to a 2-fold increase in the percentage of infected cells compared to controls for both wild type (S.A.AR86) and lab-adapted (HRsp) Sindbis virus strains but had no affect on VSV infection (FIG. 8B). Again, this was during the first round of infection because the wild type strain is a replicon unable to spread. When NRAMP2 over-expressing cells were treated with iron, a decrease in NRAMP2 expression was observed (FIG. 7A). These cells were used to determine if NRAMP2 mediated binding of Sindbis virus to mammalian cells. Using biotinylated Sindbis virus, a ˜17-fold reduction in virus binding to iron-treated cells compared to controls was observed when visualized bound virus using the microscopy-based assay (FIG. 8C, FIG. 7E). Iron-treatment had no affect on VSV binding using the same assay (FIG. 8C, FIG. 7E). Furthermore, biochemically monitored binding by immunoprecipitating bound virus using streptavidin beads could specifically detect Sindbis virus (FIG. 8E, left panel). Increased binding to NRAMP2-overexpressing cells was observed, and this binding was attenuated by iron treatment of both wild type cells and cells over-expressing NRAMP2 (FIG. 8E, right panel). Cells were bound with either biotinylated Sindbis virus, biotinylated VSV or biotinylated IgG and the bound proteins using streptavidin beads were precipitated and an immunoblot was performed. NRAMP2, but not GAPDH, was found to specifically co-precipitate with Sindbis virus while the VSV and IgG did not (FIG. 9). Furthermore, this interaction is lost under high iron concentrations which significantly deplete NRAMP2 from cells (FIG. 9). Taken together, these data demonstrate that Sindbis virus binding to mammalian cells is mediated by NRAMP2.

### Example 8

### NRAMP2 Requirement for Sindbis Infection of Mammalian Cells

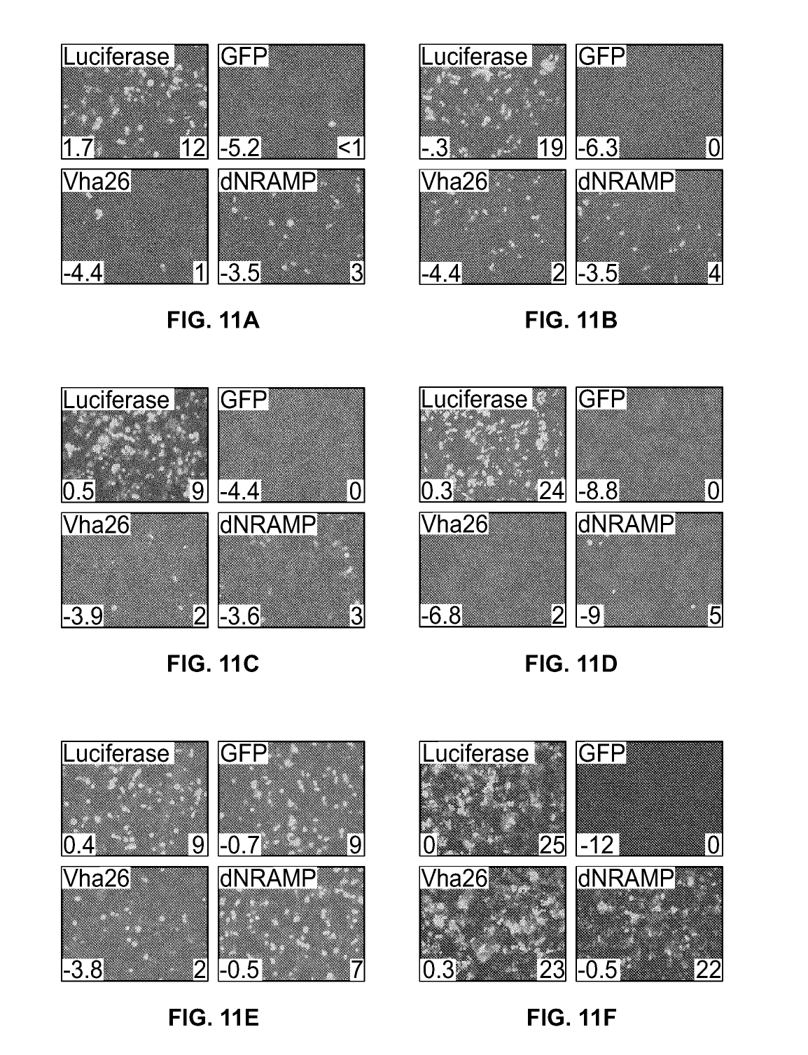

Next, it was sought to determine if NRAMP2 is required for Sindbis infection of mammalian cells. As there are no known cell lines refractive to Sindbis virus infection, and NRAMP2 knockout mice are lethal, SV40 T-antigen transformed MEFs from mice carrying floxed NRAMP2 alleles (NRAMP2″) were generated. These cells express hNRAMP2 but not hNRAMP1 (FIG. 10A). Next, the NRAMP2″ cells were transduced with either a control retrovirus that expresses GFP (MSCV iGFP) or a retrovirus that co-expresses Cre and GFP (MSCV Cre iGFP). PCR was performed to monitor the status of the hNRAMP2 locus and found that Cre transduction led to deletion of the floxed exons in the dNRAMP2 locus (FIG. 10B). When NRAMP2 deleted MEFs were challenged with Sindbis virus (Toto 1101), there was a 50-fold decrease in infection compared to Cre negative transduced MEFs (FIG. 8F). Wild type Sindbis virus (A.R.AR86, TR339) infection using these replicons was also dependent upon NRAMP2 demonstrating the requirement in the first round of infection (FIG. 8F). In contrast, loss of NRAMP2 had no effect on West Nile virus or VSV infection (FIG. 8F).

To underscore the specificity of NRAMP2 as a receptor for Sindbis virus, we tested whether Ross River virus, another alphavirus, is dependent upon NRAMP for infection. We found that Ross River virus infection is both insensitive to iron treatment (not shown) and NRAMP2 deletion (FIG. 8G). This allowed advantage to be taken of Sindbis virus/Ross River virus chimeras, in which the structural and nonstructural proteins are swapped, to demonstrate that NRAMP2 is specifically required for entry and not downstream events in the Sindbis virus life cycle. Viruses containing Sindbis virus structural proteins with either the Sindbis virus or Ross River virus nonstructural proteins were NRAMP2-dependent while viruses containing the Ross River virus structural proteins with either the Sindbis virus or Ross River virus nonstructural proteins were NRAMP2-independent (FIG. 8G). Taken together, these data show that NRAMP2 is required for Sindbis virus binding, entry and infection in both mammalian and insect cells.

NRAMP may function as a receptor for other alphaviruses, since previous studies found that Sindbis virus can partially compete with other alphaviruses for cell surface binding. Chikungunya virus and Venezuelan Equine Encephalitis virus (VEE) were tested to determine whether they were sensitive to iron, and it was found that VEE was sensitive to this treatment (FIG. 8H). The NRAMP2^{fl/fl} cells that were transduced with either MSCV iGFP or MSCV Cre iGFP were challenged with VEE or Chikungunya virus. When NRAMP2 deleted MEFs were challenged with VEE, there was a decrease in infection compared to control transduced MEFs, while Chikungunya virus infection was unaffected (FIG. 8I). This suggests that VEE, like Sindbis virus, uses NRAMP2 as a receptor in mammalian cells. Lastly, we tested whether VEE infection of insect cells was also dependent on NRAMP and found that knock-down of dNRAMP significantly attenuated infection of VEE infection of Drosophila cells (FIG. 8J). Therefore, VEE, like Sindbis virus uses NRAMP for entry both in insect and mammalian hosts.

The identification of NRAMP as a cellular receptor for the arbovirus Sindbis in both the insect and vertebrate host sheds light on the mechanism of entry and the conservation of receptor binding across diverse species. The ubiquitous expression of NRAMP and its trafficking via the clathrin-dependent endocytic pathway for delivery into acidified compartments fills two requirements for a Sindbis virus receptor.

In addition, while the overall similarity of dNRAMP with hNRAMP2 is only 54%, many of the extracellular loops have higher homology including the first extracellular loop which is 94% identical between humans and Drosophila (FIG. 6C). In particular, the most conserved extracellular loop contains the iron binding site, and iron uptake is essential for all organisms, therefore Sindbis virus may bind to this region making it difficult for organisms to escape from Sindbis virus interactions.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for treating a disease or disorder associated with an alphavirus infection in a subject, the method comprising: administering to said subject an effective amount of a molecule that inhibits the activity of a Natural Resistance-Associated Macrophage Protein (NRAMP), thereby in treating said disease or disorder in said subject.

2. The method of claim 1, wherein said NRAMP is NRAMP1.

3. The method of claim 1, wherein said NRAMP is NRAMP2.

4. The method of claim 1, wherein said alphavirus is a Sindbis virus.

5. The method of claim 1, wherein said alphavirus is a Venezuelan Equine Encephalitis virus.

6. The method of claim 1, wherein said disease is a Sindbis fever.

7. The method of claim 1, wherein said molecule is a nucleic acid molecule.

8. The method of claim 1, wherein said molecule is a dsRNA, miRNA, siRNA, anti-sense RNA, enzymatic RNA, aptamer, or an oligonucleotide molecule against said NRAMP.

9. The method of claim 1, wherein said molecule is a protein or peptide molecule.

10. The method of claim 1, wherein said molecule is an antibody that binds specifically to said NRAMP.

11. The method of claim 1, wherein said molecule is a small molecule compound.

12. The method of claim 1, wherein said molecule is an iron containing compound.

13. A method for identifying a molecule that treats a disease or disorder associated with an alphavirus infection, the method comprising contacting a cell that expresses a Natural Resistance-Associated Macrophage Protein (NRAMP) with a candidate molecule, and comparing the biological activity of the NRAMP in the cell contacted by the candidate molecule with the level of biological activity in a control cell not contacted by the candidate molecule, wherein an alteration in the biological activity of the NRAMP identifies the candidate molecule as a candidate molecule that treats a disease or disorder associated with said alphavirus infection.

14. The method of claim 13, wherein said NRAMP is NRAMP1.

15. The method of claim 13, wherein said NRAMP is NRAMP2.

16. The method of claim 13, wherein said molecule is a dsRNA, miRNA, siRNA, anti-sense RNA, enzymatic RNA, aptamer, or an oligonucleotide molecule against said NRAMP.

17. The method of claim 13, wherein said molecule is a protein or peptide molecule.

18. The method of claim 13, wherein said molecule is an antibody that binds specifically to said NRAMP.

19. The method of claim 13, wherein said molecule is a small molecule compound.

20. The method of claim 13, wherein said alphavirus is a Sindbis virus.

21. The method of claim 13, wherein said alphavirus is a Venezuelan Equine Encephalitis virus.

22. The method of claim 13, wherein said disease is a Sindbis fever.

23. A composition comprising: a molecule that inhibits the activity of a Natural Resistance-Associated Macrophage Protein (NRAMP), wherein said molecule is present in an amount effective to treat a disease or disorder associated with an alphavirus infection.

24. The composition of claim 23, wherein said NRAMP is NRAMP1.

25. The composition of claim 23, wherein said NRAMP is NRAMP2.

26. The composition of claim 23, wherein said disease is a Sindbis fever.

27. The composition of claim 23, wherein said molecule is a nucleic acid molecule.

28. The composition of claim 23, wherein said molecule is a dsRNA, miRNA, siRNA, anti-sense RNA, enzymatic RNA, aptamer, or an oligonucleotide molecule against said NRAMP.

29. The composition of claim 23, wherein said molecule is a protein or peptide molecule.

30. The composition of claim 23, wherein said molecule is an antibody that binds specifically to said NRAMP.

31. The composition of claim 23, wherein said molecule is a small molecule compound.

32. The composition of claim 23, wherein said molecule is an iron containing compound.

33. A vaccine comprising the composition of claim 23.

34. A method for predicting a risk for a disease or disorder associated with an alphavirus infection, in a subject, the method comprising: testing a sample from said to detect the presence or absence of a Natural Resistance-Associated Macrophage Protein (NRAMP), wherein the presence of said NRAMP indicates the risk for said disease or disorder, thereby predicting said risk in said subject.

35. The method of claim 34, further comprising the step of obtaining said biological sample from said subject.

36. The method of claim 34, wherein said NRAMP is NRAMP1.

37. The method of claim 34, wherein said NRAMP is NRAMP2.

38. The method of claim 34, wherein said disease is a Sindbis fever.

39. A method for increasing the expression of Natural Resistance-Associated Macrophage Protein (NRAMP) or fragment thereof in a subject and/or cell, comprising: administering to the subject and/or cell a nucleic acid encoding said NRAMP or fragment thereof, thereby enhancing the ability of an alphavirus to infect said subject and/or cell.

40. The method of claim 39, wherein said NRAMP is NRAMP1.

41. The method of claim 39, wherein said NRAMP is NRAMP2.

42. The method of claim 34, wherein the ability of an alphavirus to infect said subject and/or cell is enhanced by at least two fold.

43. The method of claim 34, wherein said fragment comprises the first extracellular loop of NRAMP.

44. The method of claim 34 further comprising the step of administering an alphavirus based gene-therapy or vaccine.


SEQUENCE LISTING (15 sequences)

SEQ ID NO: 1 - DNA, 24 bp, Homo sapiens
tcaaacttctctgggtgctgctct

SEQ ID NO: 2 - DNA, 24 bp, Homo sapiens
agagcagattgaatgcaatggccg

SEQ ID NO: 3 - DNA, 24 bp, Homo sapiens
aggacatgcccagtgcaatcaaac

SEQ ID NO: 4 - DNA, 24 bp, Homo sapiens
ttactttggccctgctgcactcta

SEQ ID NO: 5 - DNA, 24 bp, Mus musculus
tgttgcctctttgcttgctggatg

SEQ ID NO: 6 - DNA, 24 bp, Mus musculus
atgagcaaagccatcacttcgtgc

SEQ ID NO: 7 - DNA, 24 bp, Mus musculus
cgacaaatggcctttgctgtctgt

SEQ ID NO: 8 - DNA, 24 bp, Mus musculus
atatagggttgcagatgggtggca

SEQ ID NO: 9 - DNA, 20 bp, Mus musculus
atgggcgagttagaggcttt

SEQ ID NO: 10 - DNA, 20 bp, Mus musculus
cctgcatgtcagaaccaatg

SEQ ID NO: 11 - DNA, 19 bp, Mus musculus
tagctttcctggtgtttgg

SEQ ID NO: 12 - DNA, 20 bp, Mus musculus
gagcagcccagaagtaggtg

SEQ ID NO: 13 - DNA, 20 bp, Mus musculus
atttggtggtaaacactggc

SEQ ID NO: 14 - DNA, 78 bp, Drosophila melanogaster
taatacgactcactataggtggacagcaggtatatggcaaattaaccctcactaaaggtagcagcatgtggaaactcg

SEQ ID NO: 15 - DNA, 81 bp, Drosophila melanogaster
taatacgactcactataggaaagacaagtcgatgtacgaataatacgactcactataggttacagtccattaacaaactcg